(19) United States
(12) Patent Application Publication
(10) Pub. No.: US 2021/0322366 A1
(43) Pub. Date: Oct. 21, 2021

(54) HERBAL PREPARATION-ENRICHED CANNABINOID COMPOSITION AND METHODS OF TREATMENT

(71) Applicant: BUZZELET DEVELOPMENT AND TECHNOLOGIES LTD., OR-AKIVA (IL)

(72) Inventors: NOA RAZ, GIZO (IL); DANA BERNEMAN ZEITOUNI, KFAR YONA (IL); AHARON EYAL, JERUSALEM (IL)

(21) Appl. No.: 17/359,567

(22) Filed: Jun. 27, 2021

Related U.S. Application Data
(63) Continuation-in-part of application No. PCT/IB2019/061354, filed on Dec. 25, 2019.
(60) Provisional application No. 62/785,264, filed on Dec. 27, 2018.
(60) Provisional application No. 62/830,471, filed on Apr. 7, 2019.

(51) Int. Cl. A61K 31/352; A61K 31/05; A61K 45/06; A61K 36/73; A61K 31/015

(52) U.S. Cl. CPC A61K 31/352; A61K 31/05; A61K 31/015; A61K 36/73; A61K 45/06

(57) ABSTRACT

A method of treatment including administering to a subject in need a therapeutically effective amount of a product having (i) at least one cannabinoid, (ii) at least one herbal preparation, where the herbal preparation is produced by at least one of distilling and extracting a portion of a plant, where the plant is not Cannabis sativa, (iii) at least one primary terpene selected from the group consisting of caryophyllene, linalool, pinene, eucalyptol, limonene, guaiol, geraniol, sabinene, terpineol, bisabolol, humulene, myrcene, nerolidol and combinations thereof, (iv) optionally at least three secondary terpenes, and (v) at least 5% by weight of a non-cannabinoid, non-terpene carrier.

### FIELD

The field of art to which this invention generally pertains is herbal prepartion-enriched products, and specifically to herbal prepartion-enriched products for therapeutic use.

### BACKGROUND

Herbal preparations are commonly used for treating various ailments. Herbal medicine involves the medicinal use of plants to treat disease and enhance general health and wellbeing.

Cannabis is a complex plant comprising over 400 chemical entities of which more than 60 are cannabinoid compounds, some of which have opposing pharmacological effects. In the naturally occurring Cannabis plant, cannabinoids are synthesized and accumulated as cannabinoid acids (e.g. tetrahydrocannabinolic acid (THCa) and cannabidioolic acid (CBDA). When the herbal product is dried, stored and heated, the acids decarboxylize into their active forms, such as tetrahydrocannabinol (THC) and Cannabidiol (CBD) [De Meijer et al. 2003; The inheritance of chemical phenotype in Cannabis sativa L. Genetics 63: 335-346]. There are major differences in functional pharmaceutical effects between the acid and decarboxylized forms (for instance THC is psychoactive, while THCa is not psychoactive), in physical properties (such as solubility and boiling point) and in chemical properties (e.g. THCa may undergo esterification and dimerization, while THC cannot, THCa solubility is pH dependent, while that of THC is not, etc.) (e.g. De Zeeuw, Journal of Pharmacy and Pharmacology, 1972; Mechoulam, Naturwissenschaften, 1978; Grotenhermen, Clinical Pharmacokinetics, 2003).

Terpenes are the major components of the essential oils present in various Cannabis varieties. These compounds are responsible for the distinctive aromas and flavors. Terpene yield and distribution in the plant vary according to numerous parameters, such as environmental conditions or maturity of the plant. (Meier and Mediavilla, 1998; Brenneisen, 2007). Terpene yield is less than 1% in most Cannabis asays (Potter, 2009). Monoterpenes usually predominate in the natural plant (limonene, myrcene, pinene), but these headspace volatiles (Hood et al., 1973) suffer diminished yields with drying and storage (Turner et al., 1980; Ross and ElSohly, 1996), resulting in a higher relative proportion of sesquiterpenoids in processed Cannabis. Being volatile agents, terpenes futher evaporate during heating and decarboxyalation.

Terpenes play important roles in cannabinoids-comprising products, affecting the functionality and bioavailability of the cannabinoids and the aroma of the product. Processing Cannabis plant material typically leads to terpenes loss so that most of Cannabis products are of relatively low terpene content. That is particularly true for Cannabis extracts and products thereof, such as Cannabis tablets, Cannabis gel capsules, Cannabis patches, Cannabis suppositories, etc. Most Cannabis terpenes boiling points are in the range between about 150° C. and about 220° C. and they evaporate, at least partially, during Cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups.

### SUMMARY

According to an aspect of some embodiments of the present invention, there is provided a method of treatment comprising administering to a subject in need a therapeutically effective amount of a product comprising:

(i) at least one cannabinoid;
(ii) at least one herbal preparation, wherein said herbal preparation is produced by at least one of distilling and extracting a portion of a plant, wherein said plant is not Cannabis sativa;
(iii) at least one primary terpene selected from the group consisting of caryophyllene, linalool, pinene, eucalyptol, limonene, guaiol, geraniol, sabinene, terpineol, bisabolol, humulene, myrcene, nerolidol and combinations thereof;
(iv) optionally at least three secondary terpenes; and
(v) at least 5% by weight of a non-cannabinoid, non-terpene carrier.

According to some embodiments, said primary terpene is present in said product at a concentration of at least 5% of a concentration of said herbal preparation.

According to some embodiments, said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, origanum, Paeonia lactiflora, Panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, Vibronum, Viburnum opulus, Viburnum prunifolium, Viscum Albumvitex, Vitex agnus castus, Witania, Zanthoxylum spp, Zingiber officinalis and combinations thereof.

According to some embodiments, said product comprises a homogenous preparation of Cannabis flowers and herbal preparation.

According to some embodiments, the treatment is of at least one condition and/or symptom associated therewith, wherein said condition is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

According to some embodiments, said at least one cannabinoid comprises tetrahydrocannabinol (THC) at a concentration of less than 5% by weight, and at least 0.5% by weight herbal preparation.

According to some embodiments, said at least one herbal preparation is selected from the group consisting of Valeriana officinalis, Avena sativa, Passiflora incarnate, Humulus Lopulus, Bacopa monnieri, Hypericum perforatum, Lavendula officinalis, Matricaria recutita, Melissa officinalis, Piscidia erythrina, Scutellaria lateriflora, Ziziphus jujuba, Zizyphus spinosa, Lactuca spp. and combinations thereof, and wherein said treatment is of a sleep disorder or insomnia.

According to some embodiments, said at least one herbal preparation is selected from the group consisting of Hypericum Perfortum, Passiflora, Humulus Lopus, Whitania and Valeriana officinalis, Bacopa monnieri, Avena sativa, Centella (Hydrocotyl) asiatica, Cypripedium pubescens, Ganoderma lucidum, Lactuca spp. Leonurus spp. Melissa officinalis Rhodiola rosea, Scutellaria lateriflora, Tilia europea, Turnera diffusa, Ziziphus jujuba, Zizyphus spinosa and wherein the treatment is of a condition selected from the group consisting of anxiety, nervousness, restlessness, stress, mood swing, affect disorder and combinations thereof.

According to some embodiments, said at least one herbal preparation is selected from the group consisting of Boswellia spp, Curcuma longa, Origanum and Glycyrrhiza glabra, Filipendula ulmaria, Matricaria recutita, Angelica archangelica, Foeniculum vulgare, Mentha piperita, Zingiber officinale, Glycyrrhiza glabra, Angelica archangelica and combinations thereof, and wherein said treatment is of a condition selected from the group consisting of an intestinal disorder, gastrointestinal pain and a combination thereof.

According to some embodiments, said at least one herbal preparation is selected from the group consisting of Angelica sinesis, Piscidia, Vibrunum opulus, Vitex agnus castus, Dioscorea villosa, Cimicifuga racemosa, Trifolium pretense, Capsella bursa pastoris, Achillea millefolium, Boswellia spp, Paeonia lactiflora, and combinations thereof, and wherein said treatment is of pelvic pain.

According to some embodiments, said at least one herbal preparation is selected from the group consisting of Viburnum prunifolium, Zanthoxylum spp, Valeriana officinalis, Rosmarinus officinalis, Capsicum spp, Mentha piperita, Corydalis yanhusou, Paeonia lactiflora and combinations thereof, and wherein said treatment is of a condition selected from the group consisting of spasms, muscle cramps and a combination thereof.

According to some embodiments, said at least one herbal preparation is selected from the group consisting of Curcuma longa, origanum, Filipendula ulmaria, Glycyrrhiza glabra, Phelodendron amurense, Boswellia spp, Matricaria recutita, Mentha piperita, Capsella bursa pastoris, Agrimonia eupatoria, Verbena officinalis, Ganoderma lucidum, Citri reticulata, Zingiber officinale and combinations thereof, and wherein said treatment is of an inflammatory bowel disease.

According to some embodiments, said at least one herbal preparation is selected from the group consisting of Anemarrhenae asphodeloides, Cimicifuga racemosa, Salvia officinalis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa and combinations thereof, and wherein said treatment is of hot flashes.

According to some embodiments, said at least one herbal preparation is selected from the group consisting of Vitex agnus castus, Cimicifuga racemosa, Angelica sinesis and combinations thereof, and wherein said treatment is of Polycystic Ovary Syndrome (PCOS).

According to some embodiments, said at least one herbal preparation is selected from the group consisting of Vitex agnus castus, Rehmania glutinosa Preparataangelica sinesis, Dioscorea Villosa spp, Aulophyllum thalictroides, Chamaelirium lutea, Cimicifuga (Actaea) racemosa, Cyperus rotundus and combinations thereof, and wherein said treatment is of a condition selected from the group consisting of amenorrhea and oligomenorrhea.

According to some embodiments, said at least one herbal preparation is selected from the group consisting of Vitex agnus castus, Angelica sinesis, Cimicifuga racemosa, Withania somnifera, Chamaelirium lutea, Cyperus rotundus, Cypripedium pubescens, Leonurus spp, Curcuma Longa, Paeonia lactiflora, Iburnum prunifolium and combinations thereof, and wherein said treatment is of a condition selected from the group consisting of a hormonal dysfunction, a hormonal imbalance and a combination thereof.

According to some embodiments, said at least one herbal preparation is selected from the group consisting of Cimicifuga racemosa, Paeonia lactiflora, Viburnum opulus, Vitex agnus castusm, Dioscorea villosa, Chamaelirium luteum, Trifolium pratense, Lepidium meyenii, Serenoa serullata, Salvia officinalis, Verbena officinalis, Humulus lupulus, Leonurus cardiaca, Achillea millefolium, Capsella bursa pastoris, Agrimonia, Curcuma longa, Schisandra chinensis, Cynara scolymus, Bupleurum spp, Silybum marianum, Mentha piperita, Corydalis yanhusou, Valeriana officinalis, Glycyrrhiza glabra, Salix alba, Filipendula ulmaria, Calendula officinalis and combinations thereof, and wherein said treatment is of endometriosis.

According to some embodiments, said at least one herbal preparation is selected from the group consisting of Serenoa serrulata, Epimedium grandiflorum, Urtica dioica and combinations thereof, and wherein said treatment is of a prostate abnormality.

According to some embodiments, said at least one herbal preparation is selected from the group consisting of Humulus lupulus, Centella (Hydrocotyl) asiatica, Passiflora incarnata, Verbena officinalis, Valeriana officinalis, Bacopa monnieri, Avena sativa, Hypericum perforatum and combinations thereof, and wherein said treatment is of autism or an autism spectrum disorder.

According to some embodiments, said at least one herbal preparation is selected from the group consisting of Valeriana officinalis, Cypripedium pubescens, Passiflora incarnata, Hypericum perfuratum and combinations thereof, and wherein said treatment is of a condition selected from the group consisting of Tourette syndrome, motor tics, vocal tics and combinations thereof.

According to some embodiments, said at least one herbal preparation is selected from the group consisting of Glycyrrhiza glabra, Sambucus nigra, Scutellaria baicalensis, Ganoderma lucidum, Astragalus membranaceus, Stellaria media, Urtica dioica, Euphrasia officinalis, Ephedra sinica, Plantago spp and combinations thereof, and wherein said treatment is of an allergy.

### DETAILED DESCRIPTION

Unless indicated otherwise the term “aging” as used herein refers to getting older, e.g. reaching an age greater than 50, greater than 60, greater than 70 or greater than 80.

Unless indicated otherwise the term “child” as used herein, refers to a person under the age of 18.

Unless indicated otherwise the term “therapeutic effect applies to a child” as used herein, refers to therapeutic effect observed when the treated patient is a child.

Unless indicated otherwise the term “treatment” and/or “treating” as used herein, refers to treating, healing, relieving, decreasing, preventing and/or reducing the risk associated with, and/or improving the quality of life associated with a disease or condition, or the symptoms of such a disease or condition.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term “cannabinoid” as used herein refers to a compound that affects the endocannabinoid system. Cannabinoids are agonists or antagonists to receptors in the endocannabinoid system.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”.

As used herein, the term “cellulose” refers to cellulose, hemicellulose and their combinations. As used herein, the term “glycol” refers to any glycol, including ethylene glycol, polyethylene glycol, propylene glycol and polypropylene glycol. As used herein, the term “chlorophyll” refers to chlorophyll and degradation products thereof.

As used herein, “terpene” refers to both terpenes and terpenoids.

As used herein and unless indicated otherwise, the term “terpenes/cannabinoids (or terpenes to cannabinoids) weight/weight ratio” means the weight ratio between the combined amount of terpenes and the combined amount of cannabinoids.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches

As used herein, when a numerical value is preceded by the term “about”, the term “about” is intended to indicate +/−10% of that value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, provided is a therapeutic product comprising (i) at least one cannabinoid, and at least one herbal preparation, wherein said herbal preparation is produced by at least one of distilling and extracting a portion of a plant, wherein the plant is not Cannabis sativa. According to some embodiments, the product has an enhanced therapeutic effect compared with that of a product comprising the same cannabinoid amounts and being devoid of said herbal extract.

According to an aspect of some embodiments of the present invention, provided is a therapeutic product comprising (i) at least one cannabinoid in a specific amount, and at least one herbal preparation in a specific amount, wherein said herbal preparation is produced by at least one of distilling and extracting a portion of a plant, wherein the plant is not Cannabis sativa; wherein the product has an enhanced therapeutic effect compared with that of a product comprising the same cannabinoid amounts and 10% the amount of said herbal extract, 20% the amount of said herbal extract, 30%, 40%, 50%, 60%, 70%, 80% or 90% of said herbal extract. According to an embodiment, said product is a composition or the terms “product” and “composition” are used interchangeably. According to an embodiment, said product comprises a composition comprising (i) at least one cannabinoid in a specific amount, and at least one herbal preparation in a specific amount, wherein said herbal preparation is produced by at least one of distilling and extracting a portion of a plant, wherein said plant is not Cannabis sativa; wherein the product has an enhanced therapeutic effect compared with that of a product comprising the same cannabinoid amounts and being devoid of said herbal extract. According to an embodiment, said product comprises a composition resulting from processing a plant material.

According to an embodiment, said therapeutic product comprises at least one primary terpene in a specific amount, and optionally at least three secondary terpenes. According to an embodiment, said primary terpene specific amount is at least 5% of said herbal preparation amount. According to an embodiment, said primary terpene is present at a concentration of at least 6% of said herbal preparation amount, at least 7%, at least 8%, at least 10%, at least 15%, at least 20%, at least 25%, at least 30%, at least 35% or at least 40%.

According to an embodiment, said extracting comprises at least one of supercritical fluid extraction, microwave-assisted extraction, sonication extraction, Soxhlet extraction, marinated extraction, ultra-high-pressure extraction, accelerated solvent extraction, hot water extraction and combinations thereof.

According to an embodiment, said distilling comprises at least one of vacuum distillation, hydro-distillation, steam-distillation and combinations thereof.

According to an embodiment, the product has an enhanced therapeutic effect in treating and/or healing and/or relieving and/or decreasing, preventing and/or reducing the risk associated with, and/or improving the quality of life associated with conditions and/or symptoms associated with at least one of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problems, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof. According to an embodiment, the conditions and/or symptoms are associated with aging.

According to an embodiment, the product has an enhanced therapeutic effect in treating conditions and/or symptoms associated with aging. According to a related embodiment, the primary terpene is selected from the group consisting of caryophyllene, linalool, pinene, eucalyptol, sabinene, terpineol, bisabolol, humulene, myrcene, nerolidol and combinations thereof. According to an embodiment, the composition comprises at least 2 of the terpenes, at least 3, at least 4 or at least 5 of the terpenes. According to an embodiment, the primary terpene is selected from the group consisting of caryophyllene, linalool, pinene, terpineol, bisabolol, humulene, myrcene, nerolidol and combinations thereof. According to an embodiment, the primary terpene is selected from the group consisting of caryophyllene, linalool, sabinene and combinations thereof.

According to an embodiment, the enhanced therapeutic effect applies to a child.

According to an embodiment, the product has an enhanced therapeutic effect in treating a child. According to a related embodiment, the primary terpene is selected from the group consisting of caryophyllene, limonene, pinene, terpineol, humulene, bisabolol, guaiol and combinations thereof. According to an embodiment, the composition comprises at least 2 of the terpenes, at least 3, or all 4 of the terpenes. According to an embodiment, the primary terpene is selected from the group consisting of caryophyllene, limonene and combination thereof.

According to an embodiment, the conditions and/or symptoms are observed in a child.

According to an embodiment, the product is selected from the group consisting of tablets, gel capsules, medical patches, topicals, creams, varnishes, sublingual oils, sprays, edibles, beverages, suppositories, tampons, rectal candles, cigarettes, vaporizer liquids, nasal preparation, preparations containing micro and/or nano-emulsions, preparations containing micro and/or nano-particles and combinations thereof.

According to an embodiment, the product results in an increased and/or enhanced therapeutic effect compared with that of a product comprising the same cannabinoid and optionally primary terpene amount and being devoid of said herbal preparation. According to an embodiment, the product results in an increased and/or enhanced therapeutic effect compared with that of a product comprising the same cannabinoid and optionally primary terpene amount and 10% the amount of said herbal extract, 20% the amount of said herbal extract, 30%, 40%, 50%, 60%, 70%, 80% or 90% of said herbal extract. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, said enhanced therapeutic effect comprises shorter onset time of the therapeutic effect. According to an embodiment, the cannabinoids, herbal preparation and optionally primary terpenes are present in specific amounts, and the onset time of the therapeutic effect, as measured by methods known in the art, is at least 20% shorter than that of a product comprising the same amounts of cannabinoids and primary terpenes and being devoid of said herbal preparation, at least 30%, at least 40%, at least 50%, at least 60%, or at least 70% shorter. Shorter onset time, or differently put an earlier effect, is important particularly in cases of sublingual, edible and topical delivery and in cases where a rapid effect is desired, as in treating pain. According to an embodiment, the cannabinoids, herbal preparation and optionally primary terpenes are present in specific amounts, and the onset time of the therapeutic effect, as measured by methods known in the art, is at least 20% shorter than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% shorter.

According to an embodiment, said enhanced therapeutic effect comprises longer onset time of the therapeutic effect. According to an embodiment, the cannabinoids, herbal preparation and optionally primary terpenes are present in specific amounts, and the onset time of the therapeutic effect, as measured by methods known in the art, is at least 20% longer than that of a product comprising the same amounts of cannabinoids and primary terpenes and being devoid of said herbal preparation, at least 30%, at least 40%, at least 50%, at least 60%, or at least 70% longer. According to an embodiment, products of delayed onset time are used in combination with shorter onset time to reach a sustained release effect. According to an embodiment, the cannabinoids, herbal preparation and optionally primary terpenes are present in specific amounts, and the onset time of the therapeutic effect, as measured by methods known in the art, is at least 20% longer than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% longer.

According to an embodiment, said enhanced therapeutic effect comprises greater and/or increased magnitude of the therapeutic effect. According to an embodiment, the cannabinoids, herbal preparation and optionally primary terpenes are present in specific amounts, and the magnitude of the therapeutic effect, as measured by methods known in the art, is at least 20% greater compared with that of a product comprising the same amounts of cannabinoids and primary terpenes and being devoid of said herbal preparation, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost. According to an embodiment, the cannabinoids, herbal preparation and optionally primary terpenes are present in specific amounts, and the magnitude of the therapeutic effect, as measured by methods known in the art, is at least 20% greater than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% greater.

According to an embodiment, said enhanced therapeutic effect comprises longer and/or extended duration of the therapeutic effect. According to an embodiment, the cannabinoids, herbal preparation and optionally primary terpenes are present in specific amounts, and the duration of the therapeutic effect, as measured by methods known in the art, is at least 20% longer compared with that of a product comprising the same amounts of cannabinoids and primary terpenes and being devoid of said herbal preparation, at least 30%, at least 40%, at least 50%, at least 60%, at least 70%, at least 80%, at least 90% or at least 100% longer. According to an embodiment, the cannabinoids, herbal preparation and optionally primary terpenes are present in specific amounts, and the duration of the therapeutic effect, as measured by methods known in the art, is at least 20% longer compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% longer.

According to an embodiment, said enhanced therapeutic effect comprises smaller therapeutically effective amount of a product and/or reduced dosages of the product. According to an embodiment, the cannabinoids, herbal preparation and optionally primary terpenes are present in specific amounts, and the therapeutically effective amount of the product, as measured by methods known in the art, is at least 20% smaller compared with that of a product comprising the same amounts of cannabinoids and primary terpenes and being devoid of said herbal preparation, at least 30%, at least 40%, at least 50%, at least 60%, at least 70%, at least 80%, at least 90% or at least 100% smaller. According to an embodiment, the cannabinoids, herbal preparation and optionally primary terpenes are present in specific amounts, and the therapeutically effective amount of the product, as measured by methods known in the art, is at least 20% smaller compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises reduced secondary adverse symptoms. According to an embodiment, the cannabinoids, herbal preparation and optionally primary terpenes are present in specific amounts, and the secondary adverse symptoms, as measured by methods known in the art, are reduced by at least 20% compared with that of a product comprising the same amounts of cannabinoids and primary terpenes and being devoid of said herbal preparation by at least 30%, at least 40%, at least 50%, at least 60%, at least 70%, at least 80%, at least 90% or at least 100%. According to an embodiment, the cannabinoids, herbal preparation and optionally primary terpenes are present in specific amounts, and the secondary adverse symptoms, as measured by methods known in the art, are reduced by at least 20% compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced frequency of conditions and/or symptoms. According to an embodiment, the cannabinoids, herbal preparation and optionally primary terpenes are present in specific amounts, and the frequency of the conditions and/or symptoms, as measured by methods known in the art, is at least 20% smaller compared with that of a product comprising the same amounts of cannabinoids and primary terpenes and being devoid of said herbal preparation, at least 30%, at least 40%, at least 50% at least 60%, at least 70%, at least 80%, or at least 90% smaller. According to an embodiment, the cannabinoids, herbal preparation and optionally primary terpenes are present in specific amounts, and the frequency of the conditions and/or symptoms, as measured by methods known in the art is at least 20% smaller compared with that of a product amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced severity of conditions and/or symptoms. According to an embodiment, the cannabinoids, herbal preparation and optionally primary terpenes are present in specific amounts, and the severity of the conditions and/or symptoms, as measured by methods known in the art, is at least 20% smaller compared with that of a product comprising the same amounts of cannabinoids and primary terpenes and being devoid of said herbal preparation, at least 30%, at least 40%, at least 50% at least 60%, at least 70%, at least 80%, or at least 90% smaller. According to an embodiment, the cannabinoids, herbal preparation and optionally primary terpenes are present in specific amounts, and the severity of the conditions and/or symptoms, as measured by methods known in the art, is at least 20% smaller compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced consumption of other drugs. According to an embodiment, the cannabinoids, herbal preparation and optionally primary terpenes are present in specific amounts, and the consumption of other drugs is reduced by at least 20% compared with that of a product comprising the same amounts of cannabinoids and primary terpenes and being devoid of said herbal preparation, reduced by at least 30%, by at least 40%, by at least 50%, by at least 60%, by at least 70%, by at least 80%, or by at least 90%. According to an embodiment, the cannabinoids, herbal preparation and optionally primary terpenes are present in specific amounts, and the consumption of other drugs is reduced by at least 20% compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, the product comprises at least two cannabinoids, at least three, at least four or at least five. According to an embodiment, the content of each cannabinoid in the product is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively), Cannabinol in acid or decarboxylated form (CBNa or CBN, respectively), Cannabicyclol in acid or decarboxylated form (CBLa or CBL, respectively).

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the product comprises THC and/or THCa. According to an embodiment, the product comprises CBD and/or CBDa. According to an embodiment, the product comprises THC and/or THCa at a content of less than 1%, less than 0.8%, less than 0.6%, less than 0.4% or less than 0.2%. According to an embodiment, the product comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the product comprises CBG and/or CBGa. According to an embodiment, the product comprises CBN and/or CBNa. According to an embodiment, the product comprises CBC and/or CBCa. According to an embodiment, the product comprises CBL and/or CBLa. According to an embodiment, the product comprises THCV and/or THCVa. According to an embodiment, the product comprises CBDV and/or CBDVa.

According to an embodiment, the product comprises at least 2% by weight carrier, at least 3%, at least 5%, at least 7%, at least 10%, at least 12%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof.

According to an embodiment, the product comprises an herbal preparation, wherein said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment, the product comprises at least one herbal preparation selected from the group consisting of Valeriana officinalis, Avena sativa, Passiflora incarnate, Humulus Lopulus, Bacopa monnieri, Hypericum perforatum, Lavendula officinalis, Matricaria recutita, Melissa officinalis, Piscidia erythrina, Scutellaria lateriflora, Ziziphus jujuba, Zizyphus spinosa, Lactuca spp. and combinations thereof, and the condition comprises a sleep disorder and/or insomnia. According to an embodiment, the product further comprises at least one primary terpene selected from the group consisting of linalool, myrcene, caryophyllene, eucalyptol, terpineol, citronellol, pinene, terpinene, nerolidol, eucalyptol, sabinene, pinene, limonene, borneol, fenchol, bisabolol, humulene, phytol, citral and combinations thereof.

According to some embodiments, the product comprises at least one terepne selected from the group consisting of pinene, terpineol, linalool, bidabolol, borneol, guaiol and combinations thereof, wherein each of said pinene, terpineol, linalool, bidabolol, borneol and guaiol, when present is independently at a concentration of less than 5% by weight of the total terpene content.

According to an embodiment, the product comprises at least one herbal preparation selected from the group consisting of Hypericum Perfortum, Passiflora, Humulus Lopus, Whitania and Valeriana officinalis, Bacopa monnieri, Avena sativa, Centella (Hydrocotyl) asiatica, Cypripedium pubescens, Ganoderma lucidum, Lactuca spp. Leonurus spp. Melissa officinalis Rhodiola rosea, Scutellaria lateriflora, Tilia europea, Turnera diffusa, Ziziphus jujuba, Zizyphus spinosa and the condition is selected from the group consisting of anxiety, nervousness, restlessness, stress, mood swing, affect disorder and combinations thereof. According to an embodiment, the product further comprises at least one primary terpene selected from the group consisting of limonene, linalool, terpineol, citronellol, eucalyptol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof.

According to an embodiment, the product comprises at least one herbal preparation selected from the group consisting of Hypericum Perfortum, Verbena Officinalis, Centella asiatica, Rhodiola rosea, Arnica montana, Arthmisia vulgaris, Avena sativa, Cola vera nitida, Eleutherococcus senticosus, Panax ginseng, Turnera diffusa and combinations thereof, and the condition comprises depression. According to an embodiment, the product further comprises at least one primary terpene selected from the group consisting of limonene, linalool, terpineol, citronellol, eucalyptol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, guaiol, phellandrene and combinations thereof.

According to an embodiment, the product comprises at least one terepne selected from the group consisting of pinene, linalool, myrcene and terpineol and combinations thereof, wherein each of said pinene, linalool, myrcene and terpineol, when present is independently at a concentration of less than 5% by weight of the total terpene content.

According to an embodiment, the product comprises at least one herbal preparation selected from the group consisting of Bacopa monnieri, Centella asiatica, Rhodiola rosea, Ginkgo biloba, Rosmarinus officinalis, Asphalentum ponjabianum-Mumio, Cinnamomum camphora, Eleutherococcus senticosus, Lepidium meyenii, Lycium barbarum, Ocimum sanctum, Zizyphus spinosa and combinations thereof, and the condition is selected from the group consisting of difficulty in concentrating, brain fog, memory loss and combinations thereof. According to an embodiment, the product further comprises at least one primary terpene selected from the group consisting of pinene, bisabolol, guaiol, terpinene, pulegone, eucalyptol, terpineol, cymene, ocimene, limonene, linalool, caryophyllene, humulene, carene and combinations thereof.

According to an embodiment, the product comprises at least one terpene selected from the group consisting of caryophyllene and/or bisabolol, wherein each of said caryophyllene and bisabolol, when present is independently at a concentration of less than 5% by weight of the total terpene content.

According to an embodiment, the product comprises at least one herbal preparation selected from the group consisting of Ganoderma lucidum, Panax ginseng, Withania somnifera, Rhodiola rosea, Bacopa monnieri, Asphalentum ponjabianum-Mumio, Arthmisia vulgaris, Astragalus membranaceus, Atractylodes macrocephala, Centella (Hydrocotyl) asiatica, Codonopsis pilosula, Cordyceps sinensis, Coriolus versicolor, Eleutherococcus senticosus, Ganoderma lucidum, Grifola frondosa, Lentinula edodes, Lepidium meyenii, Ocimum sanctum, Rosmarinus officinalis and combinations thereof, and the condition is selected from the group consisting of weakness, reduced mental or physical energy and combinations thereof. According to an embodiment, the product further comprises at least one primary terpene selected from the group consisting of pinene, terpinene, linalool, eucalyptol, myrcene, terpineol, limonene, caryophyllene, borneol and combinations thereof.

According to an embodiment, the product comprises at least one herbal preparation selected from the group consisting of Rosmarinus officinalis, Panax ginseng, Withania somnifera, Rhodiola rosea, Bacopa monnieri and combinations thereof, and the comprises fatigue. According to an embodiment, the product further comprises at least one primary terpene selected from the group consisting of pinene, terpinene, linalool, eucalyptol, myrcene, terpineol, limonene, caryophyllene, borneol and combinations thereof.

According to an embodiment, the product comprises at least one herbal preparation selected from the group consisting of Ginkgo biloba, Lycium barbarum, Rehmania glutinosa, Bupleurum spp, Polygonium (Fallopia) multiflora, Stachys betonica, Zizyphus spinosa and combinations thereof, and the condition comprises dizziness and/or defect in balance. According to an embodiment, the product further comprises at least one primary terpene selected from the group consisting of pinene, terpinene, linalool, eucalyptol, myrcene, terpineol, limonene, caryophyllene, borneol and combinations thereof.

According to some embodiments, the product comprises at least one terepne selected from the group consisting of limonene, terpineol and combinations thereof, wherein each of said limonene andr terpineol, when present is independently at a concentration of less than 5% by weight of the total terpene content.

According to an embodiment, the product comprises at least one herbal preparation selected from the group consisting of Tanacetum parthenium, Corydalis yanhusuo, Stachys betonica, Piscidia erythrina, Valeriana officinalis, Ledebouriella divaricata, Cypripedium pubescens and combinations thereof, and the condition comprises headache and/or migraine. According to an embodiment, the product further comprises at least one primary terpene selected from the group consisting of myrcene, pinene, ocimene, camphene, terpineol, linalool, caryophyllene, eucalyptol, limonene, nerolidol, fenchol, humulene, guaiol, borneol, bisabolol and combinations thereof.

According to an embodiment, the product comprises at least one terpene selected from the group consisting of linalool, terpinolene and combinations thereof, wherein each of said linalool and terpinolene when present is independently at a concentration of less than 5% by weight of the total terpene content.

According to an embodiment, the product comprises at least one herbal preparation selected from the group consisting of Mumio, Piscidia, Rosmarinus officinalis, Curcuma longa, Valeriana officinalis, Corydalis yanhusou, Salix alba, Tanacetum parthenium and combinations thereof, and the condition comprises neuropathic pain. According to an embodiment, said product further comprises at least one primary terpene selected from the group consisting of caryophyllene, linalool, eucalyptol, amyrin, borneol, pinene, terpineol, limonene, nerolidol, geraniol, myrcene, myrcene, humulene, bisabolol, guaiol and combinations thereof.

According to an embodiment, the product comprises at least one terpene selected from the group consisting of ocimene, terpinolene and combinations thereof, wherein each of said ocimene and terpinolene when present is independently at a concentration of less than 5% by weight of the total terpene content.

According to an embodiment, the product comprises at least one herbal preparation selected from the group consisting of Origanum, Matricaria recutita, Angelica archangelica, Filipendula ulmaria, Foeniculum vulgare, Mentha piperita, Zingiber officinale, Glycyrrhiza glabra, Angelica archangelica and combinations thereof, and the condition comprises abdominal pain and/or colic. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Boswellia spp, Curcuma longa, Origanum and Glycyrrhiza glabra, Filipendula ulmaria, Matricaria recutita, Angelica archangelica, Foeniculum vulgare, Mentha piperita, Zingiber officinale, Glycyrrhiza glabra, Angelica archangelica and combinations thereof, and the condition comprises intestinal disorders and/or gastrointestinal pain. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Cinnamomum zeylanicum, Schisandra chinensis, origanum, Atractylodes macrocephala, Agrimonia eupatoria, Citri reticulata, Chamaemelum nobile, Mentha piperita, Pimpinella anisum, Thymus vulgaris, Zingiber officinale and combinations thereof, and the condition comprises diarrhea. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Aloe spp., Angelica archangelica, Rhamnus purshiana, Silybum marianum, Rheum officinalis, Juglans nigra, Taraxacum officinalis Radix, Cynara scolymus), Arctium lappa, Althea officinalis, Rehmania glutinosa and combinations thereof, and the condition comprises constipation. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaiol, terpineol and combinations thereof.

According to some embodiments, the product comprises at least one terpene selected from the group consisting of borneol, humulene, terpinolene, myrcene, and combinations thereof, wherein each of said borneol, humulene, terpinolene and myrcene when present is independently at a concentration of less than 5% by weight of the total terpene content.

According to an embodiment, the product comprises at least one herbal preparation selected from the group consisting of Angelica sinesis, Piscidia, Vibrunum opulus, Vitex agnus castus, Dioscorea villosa, Cimicifuga racemosa, Trifolium pretense, Capsella bursa pastoris, Achillea millefolium, Boswellia spp, Paeonia lactiflora, and combinations thereof, and the condition comprises pelvic pain. According to an embodiment, the product further comprises at least one primary terpene selected from the group consisting of selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaoiland combinations thereof.

According to an embodiment, the product comprises at least one terpene selected from the group consisting of pinene, guaiol, linalool, borneol, humulene, terpinolene, myrcene and combinations thereof, wherein each of said pinene, guaiol, linalool, borneol, humulene, terpinolene and myrcene, when present is independently at a concentration of less than 5% by weight of the total terpene content.

According to an embodiment, the product comprises at least one herbal preparation selected from the group consisting of Boswellia spp, Curcuma longa, Salix alba, Centella asiatica, Galium aparine, Zingiber Officinalis, Zanthoxylum spp, Withania somnifera, Silybum marianum, Filipendula ulmaria, Calendula officinalis, Harpagophytum procumbens, Scutellaria baicalensis, Tanacetum parthenium, Corydalis yanhusou, Bupleurum spp, Panax ginseng and combinations thereof, and the condition is selected from the group consisting of joint pain (arthralgia), joint stiffness, arthritis, osteoarthritis and combinations thereof. According to an embodiment, the product further comprises at least one primary terpene selected from the group consisting of myrcene, linalool, caryophyllene, eucalyptol, pinene, nerolidol, sabinene, limonene, terpinene, menth-2-en-1-ol, terpineol, geraniol, guaiol, humulene, bisabolol, borneol, fenchol and combinations thereof.

According to an embodiment, the product comprises at least one primary terpene selected from the group consisting of guaiol, limonene, pinene, borneol, linalool, terpinolene and combinations thereof, wherein each of said guaiol, limonene, pinene, borneol, linalool and terpinolene, when present is independently at a concentration of less than 5% by weight of the total terpene content.

According to an embodiment, the product comprises at least one herbal preparation selected from the group consisting of Valeriana officinalis, Viburnum opulus, Scutellaria lateriflora, Rosmarinus officinalis, Capsicum spp, Mentha piperita, Corydalis yanhusou, Paeonia lactiflora and combinations thereof, and the condition is selected from the group consisting of spasticity, muscle tension and combinations thereof. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol and combinations thereof.

According to an embodiment, the product comprises at least one herbal preparation selected from the group consisting of Boswellia spp, Galium aparine, Salix alba, Piscidia erythrina, Zanthoxylum spp, Corydalis yanhusou, Valeriana officinalis, Viburnum spp, Mentha piperita, Hypericum perforatum, Scutellaria laterifolia, Verbena officinalis and combinations thereof, and the condition comprises muscle pain and/or myalgia. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol and combinations thereof.

According to an embodiment, the product comprises at least one herbal preparation selected from the group consisting of Viburnum prunifolium, Zanthoxylum spp, Valeriana officinalis, Rosmarinus officinalis, Capsicum spp, Mentha piperita, Corydalis yanhusou, Paeonia lactiflora and combinations thereof, and the condition comprises spasms and/or muscle cramps. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol, fenchol and combinations thereof.

According to an embodiment, the product at least one terpene selected from the group consisting of pinene, guaiol, linalool, borneol, terpineol and combinations thereof, wherein each of said pinene, guaiol, linalool, borneol and terpineol, when present is independently at a concentration of less than 5% by weight of the total terpene content.

According to an embodiment, the product comprises at least one herbal preparation selected from the group consisting of Viburnum prunifolium, Zanthoxylum spp, Piscidia erythrina, Boswellia spp, Capsicum spp, Valeriana officinalis, Scutellaria lateriflora, Rosmarinus officinalis, Mentha piperita, Corydalis yanhusou, Paeonia lactiflora and combinations thereof, and the condition comprises arm pain and/or leg pain. According to an embodiment, the product further comprises at least one primary terpene selected from the group consisting of linalool, terpinene, pinene, limonene, terpineol, myrcene, nerolidol and combinations thereof.

According to an embodiment, the product comprises at least one herbal preparation selected from the group consisting of Acorus calamus, Artemisia absinthium, Atractylodes macrocephala, Matricaria recutita, Zingiber officinalis, Bupleurum spp., Chamaemelum (Anthemis) nobile, Citrus reticulata, Curcuma longa, Ioscorea Villosa spp, Elettaria cardamomum, Foeniculum vulgare, Mentha piperita, Pimpinella anisum and combinations thereof, and the condition comprises nausea and/or vomiting. According to an embodiment, the product further comprises at least one primary terpene selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof.

According to an embodiment, the product comprises at least one herbal preparation selected from the group consisting of Acorus calamus, Arthmisia vulgaris, Atractylodes macrocephala, Angelica archangelica, Zingiber officinalis, Arctium lappa, Arthmisia vulgaris, Gentiana lutea spp, and combinations thereof, and the condition is selected from the group consisting of lack of appetite, anorexia, cachexia and combinations thereof. According to an embodiment, the product further comprises at least one primary terpene selected from the group consisting of linalool, citronellol, geraniol, menthol, humulene, bisabolol, borneol, fenchol, linalool, limonene, terpineol, pinene, caryophyllene, myrcene, cymene, terpinene, valencene and combinations thereof.

According to an embodiment, the product comprises at least one terpene selected from the group consisting of pinene, guaiol, linalool, myrcene, terpinolene, terpineol and combinations thereof, wherein each of said pinene, guaiol, linalool, myrcene, terpinolene and terpineol when present is independently at a concentration of less than 5% by weight of the total terpene content.

According to an embodiment, the product comprises at least one herbal preparation selected from the group consisting of Garcinia cambogia, Fucus vesiculosus, Gymnema Sylvestre, Iris versicolor, Capsicum spp, Cola vera\nitida, Coleus forskohlii, Foeniculum vulgare, Grifola frondosa Laminaria spp, Paullinia cupana and combinations thereof, and the condition comprises weight gain and/or excessive appetite. According to an embodiment, the product further comprises at least one primary terpene selected from the group consisting of eucalyptol, caryophyllene, limonene, humulene, phytol and combinations thereof.

According to an embodiment, the product comprises at least one herbal preparation selected from the group consisting of Curcuma longa, origanum, Filipendula ulmaria, Glycyrrhiza glabra, Phelodendron amurense, Boswellia spp, Matricaria recutita, Mentha piperita, Capsella bursa pastoris, Agrimonia eupatoria, Verbena officinalis, Ganoderma lucidum, Citri reticulata, Zingiber officinale and combinations thereof, and the condition comprises an inflammatory bowel disease. According to an embodiment, the product further comprises at least one primary terpene selected from the group consisting of limonene, caryophyllene, borneol, myrcene, pinene, terpinene, isopulegol, phellandrene, carene, carvacol, terpineol, humulene, linalool, nerolidol, guaiol and combinations thereof.

According to an embodiment, the product comprises at least one herbal preparation selected from the group consisting of Crataegus spp, Leonurus spp, Salvia miltiorrhiza, Ammi visnaga, Achillea millefolium, Allium sativum, Cimicifuga racemose, Olea europaea, Salvia miltiorrhiza, Tilia europea, Viburnum spp, Valeriana officinalis, Apium graveolens, Folia Taraxacum officinalis, Urtica dioica, Zea mays, Angelica sinensis, Capsicum spp, Cinnamomum zeylanicum, Eleutherococcus senticosus, Ginkgo biloba, Aesculus hippocastanum, Ruscus aculeatus and combinations thereof, and the condition comprises cardiovascular disease. According to an embodiment, the product further comprises at least one primary terpene selected from the group consisting of linalool, camphene, limonene, pinene, borneol and combinations thereof.

According to an embodiment, the product comprises at least one herbal preparation selected from the group consisting of Crataegus spp., Leonurus spp, Valeriana officinalis and combinations thereof, and the condition comprises palpitation and/or tachycardia. According to an embodiment, the product further comprises at least one primary terpene selected from the group consisting of linalool, camphene, limonene, pinene, borneol and combinations thereof.

According to an embodiment, the product comprises at least one herbal preparation selected from the group consisting of Bacopa monnieri, Ginkgo biloba, Centella asiatica, Rosmarinus officinalis, Rhodiola rosea, Rosmarinus officinalis, Curcuma longa, Eleutherococcus senticosus, Panax ginseng, Panax quinquefolius, Rhodiola rosea, Centella, Schisandra chinensis, Ganoderma lucidum, Withania somnifera and combinations thereof, and the condition comprises Alzheimer's disease. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Hypericum Perfortum, Rhodiola rosea, Valeriana officinalis, Ginkgo biloba, Viburnum spp and combinations thereof, and the condition comprises Parkinson's disease. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises Uncaria rhynchophylla, and the condition comprises Huntington's disease. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Uncaria rhynchophylla, Vibronum Hypericum Perfortum, Rhodiola rosea, Valeriana officinalis, Ginkgo biloba and combinations thereof, and the condition comprises dystonia. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Hypericum perforatum, Viburnum spp, Verbena officinalis, Withania somnifera, Valeriana officinalis, Hypericum Perfortum, Rhodiola rosea, Valeriana officinalis, Ginkgo biloba and combinations thereof, and the condition comprises multiple sclerosis. According to an embodiment, the product further comprises at least one primary terpene selected from the group consisting of caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene, geraniol and combinations thereof.

According to an embodiment, the product comprises at least one herbal preparation selected from the group consisting of Astragalus membranceous, Ganoderma lucidum, Polygonum multiflorum, Ligustrum lucidum, Acorus gramineus and combinations thereof, and the condition comprises myasthenia gravis. According to an embodiment, the product further comprises at least one primary terpene selected from the group consisting of pinene, terpineol, eucalyptol, pulegone, cymene, ocimene, nerolidol, limonene, linalool, citronellol, geraniol, menthol, caryophyllene, borneol and combinations thereof.

According to an embodiment, the product comprises at least one herbal preparation selected from the group consisting of Bacopa monnierim, Cimicifuga racemosa, Ginkgo biloba, Paeonia lactiflora, Viscum album Uncaria rhynchophylla and combinations thereof, and the condition comprises epilepsy. According to an embodiment, the product further comprises at least one primary terpene selected from the group consisting of linalool, terpineol, ocimene, myrcene, limonene, caryophyllene and combinations thereof.

According to an embodiment, the product comprises at least one herbal preparation selected from the group consisting of Larrea mexicana, Grifola frondosa, Schisandra chinensis, Tabebuia impetiginos and Curcuma Longa, Ganoderma lucidum, Lentinula edodes, Trametes Versicolor Cordyceps sinensis, Panax ginseng, Astragalus membranaceus, Panax quinquefolius, (Silybum marianum, Scutellaria baicalensis, Boswellia spp, Glycyrrhiza glabra and combinations thereof, and the condition comprises cancer and/or cancer-related symptoms. According to an embodiment, the product further comprises at least one primary terpene selected from the group consisting of myrcene, limonene, caryophyllene, caryophyllene oxide, terpinene, citronellol, linalool, geraniol, menthol, humulene, amyrin, cycloartenol, pinene, camphene, eucalyptol, terpineol, borneol, bisabolol. nerolidol, fenchol, guaiol and combinations thereof.

According to an embodiment, the product comprises at least one terpene selected from the group consisting of pinene, guaiol, linalool, myrcene, terpinolene, terpineol and combinations thereof, wherein each of said pinene, guaiol, linalool, myrcene, terpinolene and terpineol, when present is independently at a concentration of less than 5% by weight of the total terpene content.

According to an embodiment, the product comprises at least one herbal preparation selected from the group consisting of Ginkgo Biloba, Uncaria rhynchophylla and combinations thereof, and the therapeutic effect comprises stroke and/or traumatic brain injury. According to an embodiment, the product further comprises at least one primary terpene selected from the group consisting of myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol, carotol and combinations thereof.

According to an embodiment, the product comprises at least one herbal preparation selected from the group consisting of Albizia lebbeck, Glycyrrhiza glabra, Sambucus nigra, Scutellaria baicalensis, Sophora flavescens, Echinacea spp, Inula helenium, Thymus vulgaris, Thuja occidentalis, Marrubium vulgare, Angelica archangelica, Drosera rotundifolia and combinations thereof, and the condition comprises an inflammatory airway disease. According to an embodiment, the product further comprises at least one primary terpene selected from the group consisting of pinene, eucalyptol, limonene, humulene, camphene, caryophyllene and combinations thereof.

According to an embodiment, the product comprises at least one herbal preparation selected from the group consisting of Echinacea spp., Ganoderma lucidum, Astragalus membranaceus, Rhodiola rosea, Panax ginseng, Eleutherococcus senticosus, Panax quinquefolius, Withania somnifera, Centella asiatica and combinations thereof, and the condition comprises a weakened immune system. According to an embodiment, product further comprises at least one primary terpene selected from the group consisting of linalool, pinene, borneol, caryophyllene, geraniol, amyrin, terpinene, myrcene, eucalyptol, humulene and combinations thereof.

According to an embodiment, the product comprises at least one herbal preparation selected from the group consisting of Curcuma Longa, Glycyrrhiza glabra Boswellia spp and combinations thereof, and the condition comprises inflammation. According to an embodiment, the product further comprises at least one primary terpene selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, menth-2-en-1-ol, carotol, nerolidol and combinations thereof.

According to an embodiment, the product comprises at least one herbal preparation selected from the group consisting of Momordica charantia, Gymnema Sylvestre, Trigonella foenum graecum, Ganoderma lucidum, Panax quinquefolium, Panax ginseng, Withania somnifera, Astragalus membranaceus, Schisandra chinensis, Stevia rebaudiana, Curcuma longa, Ocimum sanctum, Silybum marianum, Olea europaea, Rehmania glutinosa, Eleutherococcus senticosus and combinations thereof, and the condition comprises diabetes. According to an embodiment, the product further comprises at least one primary terpene selected from the group consisting of limonene, pinene, linalool, humulene, myrcene, phytol, geraniol, bisabolol, borneol, guaiol, ocimene, fenchol, terpineol, terpinene, eucalyptol and combinations thereof.

According to an embodiment, the product comprises at least one herbal preparation selected from the group consisting of Boswellia spp, Galium aparine, Salix alba, Piscidia erythrina, Zanthoxylum spp, Corydalis yanhusou, Valeriana officinalis, Viburnum spp, Astragalus membranaceus, Glycyrrhiza glabra, Withania somnifera, Codonopsis pilosula, Eleutherococcus senticosus, Panax ginseng, Ganoderma lucidum, Centella asiatica, Schisandra chinensis, Atractylodes macrocephala, Lepidium meyenii, Rhodiola rosea, Hypericum perforatum, Scutellaria laterifolia, Verbena officinalis and combinations thereof, and the condition comprises fibromyalgia. According to an embodiment, the product further comprises at least one primary terpene selected from the group consisting of linalool, caryophyllene, limonene, humulene, eucalyptol, amyrin, cycloartenol, pinene, sabinene, myrcene, terpinene, borneol, bisabolol and combinations thereof.

According to an embodiment, the product comprises at least one herbal preparation selected from the group consisting of Anemarrhenae asphodeloides, Cimicifuga racemosa, Salvia officinalis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa and combinations thereof, and the condition comprises hot flashes. According to an embodiment, the product further comprises at least one primary terpene selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof.

According to an embodiment, the product comprises at least one herbal preparation selected from the group consisting of Anemarrhenae asphodeloides, Salvia officinalis, Schisandra chinensis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa and the condition comprises facial flushing and/or night sweat. According to an embodiment, the product further comprises at least one primary terpene selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol, menthol and combinations thereof.

According to an embodiment, the product comprises at least one herbal preparation selected from the group consisting of Apium graveolens, Barosma betulina, Hydrastis canadensis, Plantago spp, Equisitium arvense, Urtica dioica, Zea mays, Echinacea spp, Tabebuia impetiginosa, Phellodendron spp, Arctostaphylos uva ursi, Taraxacum officinalis folia, Capsella bursa pastoris, Phyllantus spp, Scutellaria baicalensis, Thuja occidentalis, Camellia sinensis, Althea officinalis, Glycyrrhiza glabra, Plantago spp and combinations thereof, and the condition comprises a lower urinary tract disorder.

According to an embodiment, the product comprises at least one herbal preparation selected from the group consisting of Withania somnifera, Vitex agnus castus, Dioscorea Villosa spp, Asparagus racemosa, Rehmania glutinosa, Angelica sinensis, Glycyrrhiza glabra, Trifolium pratense, Humulus lupulus, Medicago sativa, Cyperus rotundus and combinations thereof, and the condition comprises vaginal dryness.

According to an embodiment, the product comprises at least one herbal preparation selected from the group consisting of Bupleurum spp, Coptis chinensis, Echinacea spp, Cimicifuga racemosa, Anemarrhenae asphodeloides, Phytolacca americana, Tabebuia impetiginosa, Uncaria tormentosa, Andrographis paniculate, Berberis vulgaris, Hydrastis canadensis, Thymus vulgaris, Boswellia spp, Glycyrrhiza glabra, Curcuma longa, Achillea millefolium, Cimicifuga racemosa, Salix alba, Filipendula ulmaria, Calendula officinalis, Scutellaria baicalensis, Zingiber officinale, Tanacetum parthenium, Corydalis yanhusou, Valeriana officinalis, Viburnum spp, Galium aparine, Phytolacca americana, Iris versicolor, Calendula, Urtica, Zea mays and combinations thereof, and the condition comprises an infection of the urogenital or reproductive system.

According to an embodiment, the product comprises at least one herbal preparation selected from the group consisting of Caulophyllum thalictroides, Viburnum prunifolium, Vitex agnus castus, Cimicifuga racemosa, Angelica sinensis, Trifolium pratense, Lepidium meyenii, Salvia officinalis, Verbena officinalis, Corydalis yanhusou, Valeriana officinalis, Achillea millefolium, Capsella bursa pastoris, Agrimonia eupatoria, Calendula officinalis, Paeonia lactiflora and combinations thereof, and the condition is selected from the group consisting of fibroids, myomas and/or cysts in the female genital system and combinations thereof.

According to an embodiment, the product comprises said product comprises at least one primary terpene selected from the group consisting of pinene, limonene, eucalyptol, caryophyllene myrcene, citronellol, humulene, terpinene, terpineol, guaiol, friedelin, eudesmol linalool, borneol, amyrin, cycloartenol, camphor, menthol and combinations thereof.

According to an embodiment, the product comprises at least one herbal preparation selected from the group consisting of Withania somnifera, Vitex agnus castus, Lepidium meyenii, Asparagus racemosa, Asphalentum ponjabianum-Mumio, Emblica officinalis Phylanthus emblica, Epimedium grandiflorum, Ganoderma lucidum, Panax ginseng, Rhodiola rosea, Tribulus terrestris, Turnera diffusa and combinations thereof, and the condition comprises reduced libido and/or sexual dysfunction.

According to an embodiment, said product comprises at least one primary terpene selected from the group consisting of limonene, linalool, myrcne, humulene, pinene, caryophyllene, bisabolol, guaiol, terpinolene, nerolidol, menthol and combinations thereof.

According to an embodiment, said product comprises at least one terpene selected from the group consisting of pinene, linalool, myrcene, terpineol and combinations thereof, wherein each of said pinene, linalool, myrcene and terpineol, when present is independently at a concentration of less than 5% by weight of the total terpene content.

According to an embodiment, the product comprises at least one herbal preparation selected from the group consisting of Vitex agnus castus, Cimicifuga racemosa, Angelica sinesis and combinations thereof, and the condition comprises Polycystic Ovary Syndrome (PCOS).

According to an embodiment, the product comprises at least one herbal preparation selected from the group consisting of Vitex agnus castus, Rehmania glutinosa Preparataangelica sinesis, Dioscorea Villosa spp, Aulophyllum thalictroides, Chamaelirium lutea, Cimicifuga (Actaea) racemosa, Cyperus rotundus and combinations thereof, and the condition is selected from the group consisting of amenorrhea and oligomenorrhea.

According to an embodiment, the product comprises at least one herbal preparation selected from the group consisting of Vitex agnus castus, Angelica sinesis, Pistacia lentiscus, Achillea millefolium, Aesculus hippocastanum, Agrimonia eupatoria, Alchemilla vulgaris, Calendula Officinalis, Capsella Bursa Pastoris, Chamaelirium lutea, Geranium maculatum, Hamamelis virginiana, Hydrastis canadensis, Pistacia lentiscus, Urtica dioica and combinations thereof, and the condition comprises hypermenorrhea.

According to an embodiment, the product comprises at least one herbal preparation is selected from the group consisting of Paeonia lactiflora, Viburnum prunifolium, Vitex Angelica sinesis, verbena officinalis, Scutalleria Laterifolia, Caulophyllum thalictroides, Chamaelirium lutea, Cimicifuga (Actaea) racemosa, Cyperus rotundus, Dioscorea Villosa spp and combinations thereof, and the condition comprises metrorrhagia.

According to an embodiment, the product comprises at least one herbal preparation selected from the group consisting of Vitex agnus castus, Angelica sinesis and combinations thereof, and the condition comprises premenstrual syndrome (PMS) and/or premenstrual dysphoric disorder (PMDD).

According to an embodiment, said product comprises at least one primary terpene selected from the group consisting of limonene, linalool, myrcne, humulene, pinene, caryophyllene, bisabolol, guaiol, terpinolene, nerolidol, menthol, camphene, citronellol, geraniol, fenchol and combinations thereof.

According to an embodiment, the product comprises at least one herbal preparation selected from the group consisting of Vitex agnus castus, Angelica sinesis, Cimicifuga racemosa, Withania somnifera, Chamaelirium lutea, Cyperus rotundus, Cypripedium pubescens, Leonurus spp, Curcuma Longa, Paeonia lactiflora, Iburnum prunifolium and combinations thereof, and the condition comprises a hormonal dysfunction and/or imbalance.

According to an embodiment, the product comprises at least one herbal preparation selected from the group consisting of Lepidium meyenii, Vitex agnus castus, Angelica sinesis, Chamaelirium lutea, Cimicifuga racemosa, Glycyrrhiza glabra, Dioscorea villosa, Trifolium pratense, Humulus lupulus, Leonurus cardiaca, Passiflora incarnata, Schisandra chinensis, Paeonia lactiflora, Angelica archangelica, Rehmania glutinosa, Urtica dioica and combination thereof, and the condition comprises a fertility disorder and/or infertility wherein the subject is a female.

According to an embodiment, the product comprises at least one herbal preparation selected from the group consisting of Chamaelirium lutea, Angelica sinesis, Cimicifuga racemosa, Tribulus terrestris, Serenoa serrulata, Lepidium meyenii, Withania somnifera, Serenoa serrulate, Ganoderma lucidum, Curcuma longa, Ginkgo biloba and combinations thereof, and the condition comprises a fertility disorder and/or infertility wherein the subject is a male.

According to an embodiment, the product comprises at least one herbal preparation selected from the group consisting of Caulophyllum thalictroides, Dioscorea Villosa spp, Rubus idaeus, Viburnum prunifolium, Panax ginseng, Zingiber officinale and combinations thereof, and the condition comprises pain associated with labour contractions.

According to an embodiment, the product comprises at least one herbal preparation selected from the group consisting of Cimicifuga racemosa, Paeonia lactiflora, Viburnum opulus, Vitex agnus castusm, Dioscorea villosa, Chamaelirium luteum, Trifolium pratense, Lepidium meyenii, Serenoa serullata, Salvia officinalis, Verbena officinalis, Humulus lupulus, Leonurus cardiaca, Achillea millefolium, Capsella bursa pastoris, Agrimonia, Curcuma longa, Schisandra chinensis, Cynara scolymus, Bupleurum spp, Silybum marianum, Mentha piperita, Corydalis yanhusou, Valeriana officinalis, Glycyrrhiza glabra, Salix alba, Filipendula ulmaria, Calendula officinalis and combinations thereof, and the condition comprises endometriosis.

According to an embodiment, said product comprises at least one primary terpene selected from the group consisting of myrcene, linalool, caryophyllene, eucalyptol, nerolidol, limonene, nerolidol, sabinene, humulene, terpinolene, guaiol, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof.

According to an embodiment, said product comprises at least one selected from the group consisting of pinene, guaiol, linalool, borneol, humulene, terpinolene, myrcene and combinations thereof, wherein said pinene, guaiol, linalool, borneol, humulene, terpinolene and myrcene are each present at a concentration of less than 5% by weight of the total terpene content.

According to an embodiment, the product comprises at least one herbal preparation selected from the group consisting of Serenoa serrulata, Epimedium grandiflorum, Urtica dioica and combinations thereof, and the condition comprises a prostate abnormality.

According to an embodiment, the product comprises at least one herbal preparation selected from the group consisting of Humulus lupulus, Centella (Hydrocotyl) asiatica, Passiflora incarnata, Verbena officinalis, Valeriana officinalis, Bacopa monnieri, Avena sativa, Hypericum perforatum and combinations thereof, and the condition comprises autism and/or an autism spectrum disorder. According to an embodiment, the product further comprises at least one primary terpene selected from the group consisting of limonene, caryophyllene, pinene, myrcene, humulene, citronellol, eucalyptol, linalool, terpinen-4-ol, ocimene, geraniol, menthol, borneol, bisabolol, fenchol, guaoil and combinations thereof.

According to an embodiment, said product comprises at least one selected from the group consisting of linalool, myrcene, terpineol and combinations thereof, wherein each of said linalool, myrcene and terpineol, when present is at a concentration of less than 5% by weight of the total terpene content.

According to an embodiment, the product comprises at least one herbal preparation is selected from the group consisting of Bacopa monnieri, Centella (Hydrocotyl) asiatica, Humulus lupulus, Valeriana officinalis, Ginco Biloba, Lactuca spp, Nepeta cataria, Scutellaria lateriflora and combinations thereof, and the condition comprises attention deficit hyperactivity disorder (ADHD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Bacopa monnieri, Withania somnifer, Ginkgo biloba, Rosmarinus officinalis, Centella asiatica, Panax ginseng, Eleutherococcus senticosus, Ginkgo biloba, Rhodiola rosea, Hypericum perforatum, Scutellaria lateriflora, Zingiber officinale and combinations thereof, and the condition comprises attention deficit disorder (ADD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Hypericum perforatum, Rhodiola rosea, Valeriana officinalis, Passiflora incarnata, Withania somnifera and combinations thereof, and the condition comprises obsessive compulsive disorder (OCD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Valeriana officinalis, Cypripedium pubescens, Passiflora incarnata, Hypericum perfuratum and combinations thereof, and the condition is selected from the group consisting of Tourette syndrome, motor tics, vocal tics and combinations thereof. According to an embodiment, the product further comprises at least one primary terpene selected from the group consisting of myrcene, linalool, limonene, pinene, nerolidol, caryophyllene, eucalyptol, geraniol, menthol, borneol, bisabolol, fenchol and combinations thereof.

According to an embodiment, the product comprises at least one herbal preparation selected from the group consisting of Drosera rotundifolia, Glycyrrhiza glabra, Plantago spp, Codonopsis pilosula, Althea officinalis, Glycyrrhiza glabra, Lactuca spp, Lonicera japonica, Lycium barbarum, Symphytum officinalis, Ulmus rubra\fulva, Verbascum Thapsus, Viola tricolor, Ziziphus jujuba and combinations thereof, and the condition comprises a dry cough. According to an embodiment, the product further comprises at least one primary terpene selected from the group consisting of pinene, eucalyptol, limonene and combinations thereof.

According to an embodiment, the product comprises at least one herbal preparation is selected from the group consisting of Thymus vulgaris, Inula helenium, Marrubium vulgare, Pimpinella anisum and combinations thereof, and the condition comprises a wet cough. According to an embodiment, the product further comprises at least one primary terpene selected from the group consisting of pinene, eucalyptol, limonene and combinations thereof.

According to an embodiment, the product comprises at least one herbal preparation selected from the group consisting of Glycyrrhiza glabra, Sambucus nigra, Scutellaria baicalensis, Ganoderma lucidum, Astragalus membranaceus, Stellaria media, Urtica dioica, Euphrasia officinalis, Ephedra sinica, Plantago spp and combinations thereof, and the condition comprises an allergy.

According to an embodiment, said product comprises at least one primary terpene selected from the group consisting of camphene, pinene, borneol, caryophyllene, geraniol and combinations thereof.

According to an embodiment, the product comprises at least one herbal preparation selected from the group consisting of Avena sativa, Lobelia inflate, Ganoderma lucidum, Withania somnifera and combinations thereof, and the condition comprises an addiction, including addiction to drugs, smoking, drinks, food, gambling, sex, and sports.

According to an embodiment, said product comprises at least one primary terpene selected from the group consisting of caryophyllene, pinene, myrcene, limonene, linalool, terpineol, citronellol, geraniol, humulene, bisabolol and combinations thereof.

According to an embodiment, the product comprises at least one herbal preparation comprise vitex and the condition comprises treatment of breast pain and/or mastalgia.

According to an embodiment, the product comprises a primary terpene and optionally at least one secondary terpenes, at least two, at least three, at least four, or at least five secondary terpenes.

As used here, the term “primary terpene” refers to a terpene that forms at least 20% by weight of the total amount of terpenes in the product, at least 25%, at least 30%, at least 35%, at least 40%, at least 50%, at least 60%, at least 70% or at least 80%. As used here, the term “primary terpene” refers to a terpene having the greatest amount in the composition. As used here, the term “primary terpene” refers to a terpene that forms the highest terpene concentration in the composition. According to an embodiment, the product comprises multiple (e.g. two, three, or four) terpenes, each one of which forms at least 20% by weight of the total amount of terpenes in the product and each one of these terpenes is considered a primary terpene. According to an embodiment, the product comprises multiple (e.g. two, three, or four) terpenes, each one of which forms at least 15% by weight of the total amount of terpenes in the product, at least 18%, at least 22%, at least 25%, at least 30%, at least 35%, or at least 40%, and each one of these terpenes is considered a primary terpene.

As used here, the term “secondary terpene” refers to a terpene that forms at least 10 parts per million (ppm) of the product. According to an embodiment, the content of the primary terpene in the product is at least 1.3 times greater than that of any secondary terpene, at least 1.5, at least 2, at least 2.2, at least 2.5, at least 3, at least 3.5, at least 4, at least 6, at least 8, at least 10, at least 15, at least 20, at least 25, or at least 30 times greater. According to an embodiment, the content of any secondary terpene in the product is at least 1.3 times smaller than that of the primary terpene, at least 1.5, at least 2, at least 2.2, at least 2.5, at least 3, at least 3.5, at least 4, at least 6, at least 8, at least 10, at least 15, at least 20, at least 25, or at least 30 times smaller.

As used herein, “herbal preparation/cannabinoids (or herbal preparation to cannabinoids) weight/weight ratio” means the weight ratio between the combined amount of herbal preparations and the combined amount of cannabinoids.

According to an embodiment, the primary terpene, the secondary terpenes or both are selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol, isomers thereof and combinations thereof. According to an embodiment, the primary terpene, the secondary terpenes or both, are selected from the group consisting of pinene, limonene, linalool, caryophyllene, myrcene, terpineol, eucalyptol, geraniol and combinations thereof. According to an embodiment, the primary terpene the secondary terpenes or both, are selected from the group consisting of pinene, limonene, linalool, caryophyllene, myrcene, terpineol, eucalyptol and combinations thereof. According to an embodiment, the primary terpene is selected from the group consisting of pinene, limonene, linalool, caryophyllene, myrcene, terpineol, eucalyptol, geraniol, humulene and combinations thereof. According to an embodiment, at least one of the terpenes is a-cyclic. According to an embodiment, at least one of the terpenes is cyclic. According to an embodiment, at least one of the terpenes is not found in Cannabis buds or is present there at less than 0.2%, less than 0.1%, less than 0.05% or less than 0.02%. Such terpene is referred to as “non-Cannabis terpene”. According to an embodiment, the terpene-enriched cannabinoid product comprises the non-Cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the terpenes comprise at least one monoterpene selected from the group consisting of limonene, myrcene, pinene, linalool, geraniol, terpinene camphene and isomers thereof. According to an embodiment, the terpenes comprise at least one sesquiterpene selected from the group consisting of nerolidol, caryophyllene, farnesene, zingiberene, vetivazulene, guaiazulene, longifolene, copaene, patchoulol humulene and isomers thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof. According to an embodiment, the terpenes comprise at least one hydroxy-terpene selected from the group consisting of nerolidol, geraniol, linalool, phytol and isomers thereof. As used herein “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, herbal preparation form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, the product comprises a composition, which is liquid at 30° C. According to an embodiment, the product comprises a composition, which is a suspension at 30° C. According to an embodiment, the product comprises a composition, which is essentially clear of haze or suspended solids at 30° C.

According to an embodiment, the product comprises Cannabis plant material. According to an embodiment, the product comprises Cannabis bud. According to an embodiment, the Cannabis bud forms at least 20% of the product, at least 30%, at least 40%, at least 50%, at least 60%, at least 70% or at least 80%.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises less than 5% by weight glycol, less than 4%, less than 3%, less than 2%, or less than 1%, by weight glycol.

According to an embodiment, the product comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the product comprises water and water content is more than 50%, more than 60%, more than 70%, more than 80%, more than 90%, or more than 95%.

According to an embodiment, the product comprises chlorophyll. According to an embodiment, the product comprises at least 0.5% chlorophyll, at least 1, % at least 5%, at least 10%, at least 15%, at least 20% chlorophyll. According to an embodiment, the product comprises ethanol. According to an embodiment, the product comprises less than about 0.5% ethanol, such as less than 0.4%, less than 0.3%, less than 0.2% or even less than 0.1% by weight ethanol. According to an embodiment, the product comprises cellulose. According to an embodiment, the product comprises less than about 0.5% cellulose, such as less than 0.4%, less than 0.3%, less than 0.2% or even less than 0.1% by weight cellulose. According to an embodiment, the product comprises at least one flavonoid. According to an embodiment, the product comprises at least two, at least three, at least four, or at least five flavonoids. According to an embodiment, the product comprises at least one selected from the group consisting of bergamottin, apigenin, amentoflavone, quercetin, piperine and combinations thereof. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine

According to an embodiment, the product results in an increased and/or enhanced therapeutic effect compared with that of a product comprising the same amounts of cannabinoids and a smaller amount of herbal preparation, e.g. one half of that amount. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of one or more additional illness and/or ones related to administering the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, said enhanced therapeutic effect comprises shorter onset time of the therapeutic effect. According to an embodiment, the cannabinoids, herbal preparation and optionally primary and/or secondary terpenes are present in specific amounts, and the onset time of the therapeutic effect, as measured by methods known in the art, is at least 20% shorter than that of a product comprising the same amounts of cannabinoids and one half the amount of the herbal preparation, at least 30%, at least 40%, at least 50%, at least 60%, or at least 70% shorter. Shorter onset time, or differently put an earlier effect, is important particularly in cases of sublingual, edible and topical delivery and in cases where a rapid effect is desired, as in treating pain.

According to an embodiment, said enhanced therapeutic effect comprises longer onset time of the therapeutic effect. According to an embodiment, the cannabinoids, herbal preparation and optionally primary and/or secondary terpenes are present in specific amounts, and the onset time of the therapeutic effect, as measured by methods known in the art, is at least 20% longer than that of a product comprising the same amounts of cannabinoids and one half the amount of the herbal preparation, at least 30%, at least 40%, at least 50%, at least 60%, or at least 70% longer. According to an embodiment, products of delayed onset time are used in combination with shorter onset time to reach a sustained release effect.

According to an embodiment, said enhanced therapeutic effect comprises greater and/or increased magnitude of the therapeutic effect. According to an embodiment, the cannabinoids, herbal preparation and optionally primary and/or secondary terpenes are present in specific amounts, and the magnitude of the therapeutic effect, as measured by methods known in the art, is at least 20% greater compared with that of a product comprising the same amounts of cannabinoids and one half the amount of the herbal preparation, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, said enhanced therapeutic effect comprises longer and/or extended duration of the therapeutic effect. According to an embodiment, the cannabinoids, herbal preparation and optionally primary and/or secondary terpenes are present in specific amounts, and the duration of the therapeutic effect, as measured by methods known in the art, is at least 20% longer compared with that of a product comprising the same amounts of cannabinoids and one half the amount of the herbal preparation, at least 30%, at least 40%, at least 50%, at least 60%, at least 70%, at least 80%, at least 90% or at least 100% longer.

According to an embodiment, said enhanced therapeutic effect comprises smaller therapeutically effective amount of a product and/or reduced dosages of the product. According to an embodiment, the cannabinoids, herbal preparation and optionally primary and/or secondary terpenes are present in specific amounts, and the therapeutically effective amount of the product, as measured by methods known in the art, is at least 20% smaller compared with that of a product comprising the same amounts of cannabinoids and one half the amount of the herbal preparation, at least 30%, at least 40%, at least 50%, at least 60%, at least 70%, at least 80%, at least 90% or at least 100% smaller.

According to an embodiment, said enhanced therapeutic effect comprises reduced secondary adverse symptoms. According to an embodiment, the cannabinoids, herbal preparation and optionally primary and/or secondary terpenes are present in specific amounts, and the secondary adverse symptoms, as measured by methods known in the art, are reduced by at least 20% compared with that of a product comprising the same amounts of cannabinoids and one half the amount of the herbal preparation, by at least 30%, at least 40%, at least 50%, at least 60%, at least 70%, at least 80%, at least 90% or at least 100%.

According to an embodiment, said enhanced therapeutic effect comprises smaller and/or reduced frequency of conditions and/or symptoms. According to an embodiment, the cannabinoids, herbal preparation and optionally primary and/or secondary terpenes are present in specific amounts, and the frequency of the conditions and/or symptoms, as measured by methods known in the art, is at least 20% smaller compared with that of a product comprising the same amounts of cannabinoids and one half the amount of the herbal preparation, at least 30%, at least 40%, at least 50% at least 60%, at least 70%, at least 80%, or at least 90% smaller. According to an embodiment, said enhanced therapeutic effect comprises smaller and/or reduced severity of conditions and/or symptoms. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amounts of cannabinoids and secondary terpenes and one half the amount of the primary terpene, at least 30%, at least 40%, at least 50% at least 60%, at least 70%, at least 80%, or at least 90% smaller.

According to an embodiment, said enhanced therapeutic effect comprises smaller and/or reduced consumption of other drugs. According to an embodiment, the cannabinoids, herbal preparation and optionally primary and/or secondary terpenes are present in specific amounts, and the consumption of other drugs is reduced by at least 20% compared with that of a product comprising the same amounts of cannabinoids and one half the amount of the herbal preparation, reduced by at least 30%, by at least 40%, by at least 50%, by at least 60%, by at least 70%, by at least 80%, or by at least 90%.

According to an embodiment, the product comprises tetrahydrocannabinol (THC) at a concentration of less than 5% by weight, and at least 0.5% by weight primary terpene. According to an embodiment, the product is applied to a child. According to an embodiment, the primary terpene is selected from the group consisting of limonene, linalool, eucalyptol, myrcene, caryophyllene, terpineol, bisabolol, pinene and combinations thereof. According to an embodiment, the composition comprises tetrahydrocannabinol (THC) in a concentration of less than 5%, less than 4%, less than 3%, less than 2%, or less than 1%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of limonene, linalool, myrcene, caryophyllene, terpineol, bisabolol, pinene and combination thereof. According to an embodiment, the herbal preparation is in a concentration of at least 0.5%, at least 1%, at least 5%, 10%, 20%, 30%, 40%, 50%, 60%, 70%, 80% or 90%. According to an embodiment, the composition comprises at least 2 of said herbal preparation, at least 3, at least 4, or at least 5.

According to an embodiment, the product comprises CBD and optionally THC at CBD to THC weight/weight ratio greater than 1, wherein CBD concentration is less than 20% by weight and wherein primary terpene concentration is at least 0.5% by weight. According to an embodiment, the product is applied to a child. According to an embodiment, the primary terpene is selected from the group consisting of bisabolol, guaiol, linalool, terpineol, ocimene, humulene, caryophyllene, eucalyptol, pinene, limonene and combinations thereof. According to an embodiment, the product comprises CBD and optionally THC at CBD to THC weight/weight ratio greater 2, greater than 3, greater than 4 or greater than 5. According to an embodiment, the composition comprises cannabidiol (CBD) and/or cannabidiolic acid (CBDa) in a concentration of less than 18%, less than 16%, less than 14%, less than 12%, or less than 10%. According to an embodiment, the composition comprises cannabidiol (CBD) and/or cannabidiolic acid (CBDa), wherein CBD and/or CBDa concentration is of 10 to 20%, 10 to 15%, 15 to 20%, 12-18%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of linalool, terpineol, caryophyllene and combination thereof. According to an embodiment, the primary terpene is selected from the group consisting of linalool, terpineol, ocimene and combination thereof. According to an embodiment, the primary terpene is selected from the group consisting of eucalyptol, pinene, limonene and combination thereof. According to an embodiment, the primary terpene comprises caryophyllene and/or humulene. According to an embodiment, the herbal preparation is in a concentration of at least 0.5%, at least 1%, at least 5%, 10%, 20%, 30%, 40%, 50%, 60%, 70%, 80% or 90%. According to an embodiment, the composition comprises at least 2 of said herbal preparation, at least 3, at least 4, or at least 5.

According to an embodiment, the therapeutic effect is generated while administering the product in a vaporizer. According to an embodiment, the therapeutic effect is generated while administering the product by inhaling. According to an embodiment, the therapeutic effect is generated while administering the product by smoking. According to an embodiment, the therapeutic effect is generated while administering the product sublingually. According to an embodiment, the therapeutic effect is generated while applying the product, topically. According to an embodiment, the therapeutic effect is generated while administering the product in suppositories, including tampons and/or rectal candles. According to an embodiment, the therapeutic effect is generated while administering the product in a spray. According to an embodiment, the therapeutic effect is generated while administering the product as an edible, as a candy, as baked good, as a drink. According to an embodiment, the therapeutic effect is generated while administering the product as vaporizer liquid nasal preparation. According to an embodiment, the therapeutic effect is generated while administering the product as micro and nano-emulsions. According to an embodiment, the therapeutic effect is generated while administering the product as micro and nano-emulsions. According to an embodiment, the tablet is a compressed tablet. According to an embodiment, the product comprises a homogenous preparation of Cannabis flowers and said herbal preparation.

According to an embodiment, the enhanced therapeutic effect comprises reducing side effects associated with Cannabis consumption. According to an embodiment, the product comprises an additive selected from the group consisting of antioxidants, emulsifiers and texturizers, vegetable oils, plant extracts, honey, pharmaceutical excipients, sucrose, glucose and fructose, pharmaceutical excipients and combinations thereof. According to an embodiment, the product comprises a surfactant selected from the group consisting of phospholipids, glycerides, glycolipids and combinations thereof. According to an embodiment, the product further comprises a food-approved texturizer. According to an embodiment, the product further comprises at least 10 ppm ethanol. According to an embodiment, the product further comprises at least one of vitamin C, vitamin E, polyunsaturated fatty acids, beeswax and coconut oil. According to an embodiment, the product further comprises a sweetener. According to an embodiment, the product further comprises omega 3 fatty acid. According to an embodiment, the product further comprises omega 6 fatty acid. According to an embodiment, the product further comprises curcumin.

According to an embodiment, the terpene-enriched cannabinoid product of improved therapeutic effect is a human medication. According to an embodiment, the herbal preparation-enriched cannabinoid product of improved therapeutic effect is a veterinary medication.

According to an embodiment, the shelf life of the product is at least 6 months or at least a year. According to an embodiment, primary terpene degradation in the product is less than 20% per year.

According to an embodiment, further provided is a product selected from the group consisting of tablets, gel capsules, medical patches, topicals, creams, varnishes, sublingual oils, sprays, edibles, beverages, suppositories, tampons, rectal candle, cigarette, vaporizer liquid nasal preparation, preparations containing micro and nano-emulsions, preparations containing micro and nano-particles and combinations thereof, containing the product.

According to an embodiment, further provided is a commercial product comprising two products according to the present invention, which two products differ in the content of the herbal preparation. According to an embodiment, further provided is a commercial product comprising two products according to the present invention, which two products comprise different herbal preparation.

According to an embodiment, further provided is a method for producing the product, comprising providing at least one cannabinoid and blending it with an herbal preparation.

According to an embodiment, the method includes extracting Cannabis plant material to form an extract. According to an embodiment the extracting comprises steam distillation. According to an embodiment, the method further comprises removing terpenes from the extract prior to the blending. According to an embodiment, the extracting comprises contacting with an extractant to form an extract, which extract comprises at least one cannabinoid and the extractant; and optionally removing at least a fraction of the extractant from the extract. According to an embodiment, the extractant comprises at least one of ethanol, a liquefied gas, such as butane, butene or dimethyl-ether, liquefied CO₂, near-critical CO₂ supercritical CO₂ and combinations thereof. According to an embodiment, extracting comprises contacting the Cannabis plant material with an extractant to form an extract, and the extractant comprises at least one terpene. According to an embodiment, the extractant comprises the primary terpene.

According to an embodiment, the method further comprises at least partially decarboxylating the cannabinoid. According to an embodiment the decarboxylating is conducted at a temperature greater than 100° C. According to an embodiment the decarboxylating is conducted prior to extracting. According to an embodiment the decarboxylating is conducted on the extract prior to the blending with the primary terpene. According to an embodiment, a fraction of the extract is decarboxylated, and the decarboxylated fraction is blended with another fraction, which was not decarboxylated.

According to an embodiment, the method further comprises extracting a plant material, whereby the primary terpene is produced. According to an embodiment, the plant material is selected from the group including Cannabis, lemons, oranges, hops, lavender, pine needles, Echinacea, tea, clover, Capsicum, Eucalyptus, geranium and mint.

According to an embodiment, the method comprises synthesizing at least one cannabinoid and blending the synthesized cannabinoid with the primary terpene.

According to an embodiment, the method further comprises blending Cannabis plant material with the primary terpene. According to an embodiment, the Cannabis plant material comprises a Cannabis bud. According to an embodiment, the blending Cannabis plant material with the primary terpene comprises spraying the Cannabis plant material with the primary terpene, optionally in a solvent. According to an embodiment, the blending Cannabis plant material with the primary terpene comprises combining the Cannabis plant material with a substrate comprising the primary terpene. According to an embodiment, the substrate is a plant material sprayed with the primary terpene. According to an embodiment, the substrate is a cigarette paper sprayed with the primary terpene.

According to an embodiment, further provided is a method for producing the product, comprising extracting Cannabis plant material to form an extract, wherein the extracting forms at least two extract fractions, a cannabinoid-enriched extract and a terpene-enriched extract. As used herein, the term “cannabinoid-enriched extract” refers to an extract wherein cannabinoid to terpenes weight/weight ratio is greater than that in the plant material. As used herein, the term “terpene-enriched extract” refers to an extract wherein cannabinoid to terpenes weight/weight ratio is smaller than that in the plant material. According to an embodiment, the method comprises dividing the cannabinoid-enriched extract into at least two fractions and mixing at least one fraction with at least part of the terpene-enriched extract to form a terpene-enriched product, wherein terpenes to cannabinoid weight/weight ratio is greater than that ratio in the Cannabis plant material. According to an embodiment the ratio is 1.5 times greater than that in the Cannabis plant material, 2 times greater, 2.5 times greater, 3 times greater, 4 times greater, 5 times greater, 6 times greater, 7 times greater, 8 times greater, or 10 times greater. According to an embodiment, the terpene-enriched product is further mixed with at least one terpene.

According to an embodiment, provided is a method for producing said product, comprising providing at least one cannabinoid and blending it with an herbal preparation. According to an embodiment, said method comprises contacting Cannabis plant material with an extractant to form an extract and blending said extract with said herbal preparation. According to an embodiment, said method comprises synthesizing at least one cannabinoid and blending said synthesized cannabinoid with said herbal preparation. According to an embodiment, said method comprises blending Cannabis plant material with said primary terpene. According to an embodiment, said method comprises blending said product with said primary terpene.

According to an embodiment, further provided is a method of treatment comprising administering to a subject in need a therapeutically effective amount of a product comprising (i) at least one cannabinoid and (ii) at least one herbal preparation in a specific amount, wherein said herbal preparation is produced by at least one of distilling and extracting a portion of a plant, wherein the plant is not Cannabis sativa. According to some embodiments, the product provides an enhanced therapeutic effect compared with that obtained by administrating a product comprising the same cannabinoids amounts and being devoid of the herbal preparation.

According to an embodiment, further provided is a product comprising (i) at least one cannabinoid in a specific amount, and (ii) at least one herbal preparation in a specific amount, wherein said herbal preparation is produced by at least one of distilling and extracting a portion of a plant, wherein the plant is not Cannabis sativa, for use in therapy. According to some embodiments, therapy comprises treatment of a condition arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

According to an embodiment, further provided is the use of a product comprising (i) at least one cannabinoid in a specific amount, and (ii) at least one herbal preparation in a specific amount, wherein said herbal preparation is produced by at least one of distilling and extracting a portion of a plant, wherein the plant is not Cannabis sativa, for the treatment of a condition selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, Leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

According to an embodiment, provided is a therapeutic product comprising (i) at least one cannabinoid in a specific amount, and at least one herbal preparation in a specific amount, wherein said herbal preparation is produced by at least one of distilling and extracting a portion of a plant, wherein the plant is not Cannabis sativa; wherein the product has an enhanced therapeutic effect compared with that of a product comprising the same cannabinoid amounts and 10% the amount of said herbal extract, 20% the amount of said herbal extract, 30%, 40%, 50%, 60%, 70%, 80% or 90% of said herbal extract. According to an embodiment, the product comprises at least one primary terpene in a specific amount and optionally at least three secondary terpenes. According to an embodiment, said primary terpene specific amount is at least 5% of said herbal preparation amount. According to an embodiment, said primary terpene specific amount is at least 6% of said herbal preparation amount, at least 7%, at least 8%, at least 10%, at least 15%, at least 20%, at least 25%, at least 30%, at least 35% or at least 40%.

According to a related embodiment, said extracting comprises at least one of supercritical fluid extraction, microwave-assisted extraction, sonication extraction, Soxhlet extraction, marinated extraction, ultra-high pressure extraction, accelerated solvent extraction, hot water extraction and combinations thereof.

According to a related embodiment, said distilling comprises at least one of vacuum distillation, hydro-distillation, steam-distillation and combinations thereof.

According to an embodiment, the administrated product has an enhanced therapeutic effect in treating and/or healing and/or relieving and/or decreasing, preventing and/or reducing the risk associated with, and/or improving the quality of life associated with conditions and/or symptoms associated with at least one of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, Leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof. According to an embodiment, the conditions and/or symptoms are associated with aging.

According to an embodiment, the administrated product has an enhanced therapeutic effect in treating conditions and/or symptoms associated with aging. According to a related embodiment, the primary terpene is selected from the group consisting of caryophyllene, linalool, pinene, eucalyptol, sabinene, terpineol, bisabolol, humulene, myrcene, nerolidol and combinations thereof. According to an embodiment, the composition comprises at least 2 of the terpenes, at least 3, at least 4 or at least 5 of the terpenes. According to an embodiment, the primary terpene is selected from the group consisting of caryophyllene, linalool, pinene, terpineol, humulene, bisabolol, guaiol and combinations thereof. According to an embodiment, the primary terpene is selected from the group consisting of caryophyllene, linalool, sabinene and combinations thereof.

According to an embodiment, the enhanced therapeutic effect applies to a child.

According to an embodiment, the administrated product has an enhanced therapeutic effect in treating a child. According to a related embodiment, the primary terpene is selected from the group consisting of caryophyllene, limonene, pinene, terpineol and combinations thereof. According to an embodiment, the composition comprises at least 2 of the terpenes, at least 3, or all 4 of the terpenes. According to an embodiment, the primary terpene is selected from the group consisting of caryophyllene, limonene and combination thereof.

According to an embodiment, the conditions and/or symptoms are observed in a child.

According to an embodiment, the product is selected from the group consisting of cigarettes, vaporizer plant material, vaporizer liquid, extract, tablets, gel capsules, suppositories, tampons, rectal candle, cigarette, vaporizer liquid, nasal preparation, preparations containing micro and nano-emulsions, preparations containing micro and nano-particles and combinations thereof. According to an embodiment, the daily dose of the product comprises about 1 milligram cannabinoid to about 300 milligram, about 2 milligram cannabinoid to about 200 milligram or about 3 milligram cannabinoid to about 100 milligram. According to another embodiment, the daily dose of the product comprises about 0.5 milligram cannabinoid per kilogram body weight to about 30 milligram cannabinoid per kilogram body weight.

According to an embodiment, the administered product comprises at least two cannabinoids, at least three, at least four or at least five. According to an embodiment, the content of each cannabinoid in the product is at least 10 parts per million (ppm). According to an embodiment, the cannabinoids are selected from the group consisting THC, CBD, CBG, CBC, CBN, CBL, THCV, CBDV and their non-decarboxylated form thereof.

According to an embodiment, the administered product comprises at least 2% by weight carrier, at least 3%, at least 5%, at least 7%, at least 10%, at least 12%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof.

According to an embodiment, the administered product comprises an herbal preparation, wherein said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, origanum, Paeonia lactiflora, panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment, the product comprises at least one herbal preparation selected from the group consisting of Valeriana officinalis, Avena sativa, Passiflora incarnate, Humulus Lopulus, Bacopa monnieri, Hypericum perforatum, Lavendula officinalis, Matricaria recutita, Melissa officinalis, Piscidia erythrina, Scutellaria lateriflora, Ziziphus jujuba, Zizyphus spinosa, Lactuca spp. and combinations thereof and the condition comprises a sleep disorder and/or insomnia. According to an embodiment, the product further comprises at least one primary terpene selected from the group consisting of linalool, myrcene, caryophyllene, eucalyptol, terpineol, citronellol, pinene, terpinene, nerolidol, eucalyptol, sabinene, pinene, limonene, borneol, fenchol, bisabolol, humulene, phytol, citral and combinations thereof. According to an embodiment, the product comprises at least one terpene selected from the group consisting of pinene, terpineol, linalool, bidabolol, borneol, guaiol and combinations thereof, wherein each of said pinene, terpineol, linalool, bidabolol, borneol and guaiol, when present is independently at a concentration of less than 5% by weight of the total terpene content

According to an embodiment, the product comprises at least one herbal preparation selected from the group consisting of Hypericum Perfortum, Passiflora, Humulus Lopus, Whitania, Valeriana officinalis Bacopa monnieri, Avena sativa, Centella (Hydrocotyl) asiatica, Cypripedium pubescens, Ganoderma lucidum, Lactuca spp. Leonurus spp. Melissa officinalis Rhodiola rosea, Scutellaria lateriflora, Tilia europea, Turnera diffusa, Ziziphus jujuba, Zizyphus spinosa, and the condition is selected from the group consisting of anxiety, nervousness, restlessness, stress, mood swing, affect disorder and combinations thereof. According to an embodiment, the product further comprises at least one primary terpene selected from the group consisting of limonene, linalool, terpineol, citronellol, eucalyptol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene and combinations thereof.

According to an embodiment, the product comprises at least one herbal preparation selected from the group consisting of Hypericum Perfortum, Verbena Officinalis, Centella asiatica, Rhodiola rosea, Arnica montana, Arthmisia vulgaris, Avena sativa, Cola vera nitida, Eleutherococcus senticosus, Panax ginseng, Turnera diffusa and combinations thereof, and the condition comprises depression. According to an embodiment, the product further comprises at least one primary terpene selected from the group consisting of limonene, linalool, terpineol, citronellol, eucalyptol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, guaiol, phellandrene and combinations thereof.

According to an embodiment, the product comprises at least one terepne selected from the group consisting of pinene, linalool, myrcene and terpineol and combinations thereof, wherein each of said pinene, linalool, myrcene and terpineol, when present is independently at a concentration of less than 5% by weight of the total terpene content.

According to an embodiment, the product comprises at least one herbal preparation selected from the group consisting of Bacopa monnieri, Centella asiatica, Rhodiola rosea, Ginkgo biloba, Rosmarinus officinalis, Asphalentum ponjabianum-Mumio, Cinnamomum camphora, Eleutherococcus senticosus, Lepidium meyenii, Lycium barbarum, Ocimum sanctum, Zizyphus spinosa and combinations thereof, and the condition is selected from the group consisting of difficulty in concentrating, brain fog, memory loss and combinations thereof. According to an embodiment, the product further comprises at least one primary terpene selected from the group consisting of pinene, bisabolol, guaiol, terpinene, pulegone, eucalyptol, terpineol, cymene, ocimene, limonene, linalool, caryophyllene, humulene, carene and combinations thereof.

According to an embodiment, the product comprises at least one terpene selected from the group consisting of caryophyllene and/or bisabolol, wherein each of said caryophyllene and bisabolol, when present is independently at a concentration of less than 5% by weight of the total terpene content.

According to an embodiment, the product comprises at least one herbal preparation selected from the group consisting of Ganoderma lucidum, Panax ginseng, Withania somnifera, Rhodiola rosea, Bacopa monnieri, Asphalentum ponjabianum-Mumio, Arthmisia vulgaris, Astragalus membranaceus, Atractylodes macrocephala, Centella (Hydrocotyl) asiatica, Codonopsis pilosula, Cordyceps sinensis, Coriolus versicolor, Eleutherococcus senticosus, Ganoderma lucidum, Grifola frondosa, Lentinula edodes, Lepidium meyenii, Ocimum sanctum, Rosmarinus officinalis and combinations thereof, and the condition is selected from the group consisting of weakness, reduced mental or physical energy and combinations thereof. According to an embodiment, the product further comprises at least one primary terpene selected from the group consisting of pinene, terpinene, linalool, eucalyptol, myrcene, terpineol, limonene, caryophyllene, borneol and combinations thereof.

According to an embodiment, the product comprises at least one herbal preparation selected from the group consisting of Rosmarinus officinalis, Panax ginseng, Withania somnifera, Rhodiola rosea, Bacopa monnieri and combinations thereof, and the comprises fatigue. According to an embodiment, the product further comprises at least one primary terpene selected from the group consisting of pinene, terpinene, linalool, eucalyptol, myrcene, terpineol, limonene, caryophyllene, borneol and combinations thereof.

According to an embodiment, the product comprises at least one herbal preparation selected from the group consisting of Ginkgo biloba, Lycium barbarum, Rehmania glutinosa, Bupleurum spp, Polygonium (Fallopia) multiflora, Stachys betonica, Zizyphus spinosa and combinations thereof, and the condition comprises dizziness and/or defect in balance. According to an embodiment, the product further comprises at least one primary terpene selected from the group consisting of pinene, terpinene, linalool, eucalyptol, myrcene, terpineol, limonene, caryophyllene, borneol and combinations thereof.

According to an embodiment, said product comprises at least one terepne selected from the group consisting of limonene, terpineol and combinations thereof, wherein each of said limonene andr terpineol, when present is independently at a concentration of less than 5% by weight of the total terpene content.

According to an embodiment, the product comprises at least one herbal preparation selected from the group consisting of Tanacetum parthenium, Corydalis yanhusuo, Stachys betonica, Piscidia erythrina, Valeriana officinalis, Ledebouriella divaricata, Cypripedium pubescens and combinations thereof, and the condition comprises headache and/or migraine. According to an embodiment, the product further comprises at least one primary terpene selected from the group consisting of myrcene, pinene, ocimene, camphene, terpineol, linalool, caryophyllene, eucalyptol, limonene, nerolidol, fenchol, humulene, guaiol, borneol, bisabolol and combinations thereof.

According to an embodiment, said product comprises at least one terpene selected from the group consisting of linalool, terpinolene and combinations thereof, wherein each of said linalool and terpinolene when present is independently at a concentration of less than 5% by weight of the total terpene content.

According to an embodiment, the product comprises at least one herbal preparation selected from the group consisting of Mumio, Piscidia, Rosmarinus officinalis, Curcuma longa, Valeriana officinalis, Corydalis yanhusou, Salix alba, Tanacetum parthenium and combinations thereof, and the condition comprises neuropathic pain. According to an embodiment, said product further comprises at least one primary terpene selected from the group consisting of caryophyllene, linalool, eucalyptol, amyrin, borneol, pinene, terpineol, limonene, nerolidol, geraniol, myrcene, humulene, bisabolol, guaiol and combinations thereof.

According to an embodiment, said product comprises at least one terpene selected from the group consisting of ocimene, terpinolene and combinations thereof, wherein each of said ocimene and terpinolene when present is independently at a concentration of less than 5% by weight of the total terpene content.

According to an embodiment, the product comprises at least one herbal preparation selected from the group consisting of Origanum, Matricaria recutita, Angelica archangelica, Filipendula ulmaria, Foeniculum vulgare, Mentha piperita, Zingiber officinale, Glycyrrhiza glabra, Angelica archangelica and combinations thereof, and the condition comprises abdominal pain and/or colic. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Boswellia spp, Curcuma longa, Origanum and Glycyrrhiza glabra, Filipendula ulmaria, Matricaria recutita, Angelica archangelica, Foeniculum vulgare, Mentha piperita, Zingiber officinale, Glycyrrhiza glabra, Angelica archangelica and combinations thereof, and the condition comprises intestinal disorders and/or gastrointestinal pain. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Cinnamomum zeylanicum, Schisandra chinensis, origanum, Atractylodes macrocephala macrocephala, Agrimonia eupatoria, Citri reticulata, Chamaemelum nobile, Mentha piperita, Pimpinella anisum, Thymus vulgaris, Zingiber officinale and combinations thereof, and the condition comprises diarrhea. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof.

According to an embodiment, the product comprises at least one herbal preparation selected from the group consisting of Aloe spp., Angelica archangelica, Rhamnus purshiana, Silybum marianum, Rheum officinalis, Juglans nigra, Taraxacum officinalis Radix, Cynara scolymus), Arctium lappa, Althea officinalis, Rehmania glutinosa and combinations thereof, and the condition comprises constipation. According to an embodiment, the product further comprises at least one primary terpene selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaiol and combinations thereof.

According to an embodiment, said product comprises at least one terpene selected from the group consisting of borneol, humulene, terpinolene, myrcene, and combinations thereof, wherein each of said borneol, humulene, terpinolene and myrcene, when present is independently at a concentration of less than 5% by weight of the total terpene content.

According to an embodiment, the product comprises at least one herbal preparation selected from the group consisting of Angelica sinesis, Piscidia, Vibrunum opulus, Vitex agnus castus, Dioscorea villosa, Cimicifuga racemosa, Trifolium pretense, Capsella bursa pastoris, Achillea millefolium, Boswellia spp, Paeonia lactiflora, and combinations thereof, and the condition comprises pelvic pain. According to an embodiment, the product further comprises at least one primary terpene selected from the group consisting of selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaiol and combinations thereof.

According to an embodiment, said product comprises at least one terpene selected from the group consisting of pinene, guaiol, linalool, borneol, humulene, terpinolene, myrcene and combinations thereof, wherein each of said pinene, guaiol, linalool, borneol, humulene, terpinolene and myrcene, when present is independently at a concentration of less than 5% by weight of the total terpene content.

According to an embodiment, the product comprises at least one herbal preparation selected from the group consisting of Boswellia spp, Curcuma longa, Salix alba, Centella asiatica, Galium aparine, Zingiber Officinalis, Zanthoxylum spp, Withania somnifera, Silybum marianum, Filipendula ulmaria, Calendula officinalis, Harpagophytum procumbens, Scutellaria baicalensis, Tanacetum parthenium, Corydalis yanhusou, Bupleurum spp, Panax ginseng and combinations thereof, and the condition is selected from the group consisting of joint pain (arthralgia), joint stiffness, arthritis, osteoarthritis and combinations thereof. According to an embodiment, the product further comprises at least one primary terpene selected from the group consisting of myrcene, linalool, caryophyllene, eucalyptol, pinene, nerolidol, sabinene, limonene, terpinene, menth-2-en-1-ol, terpineol, geraniol, guaiol, humulene, bisabolol, borneol, fenchol and combinations thereof.

According to an embodiment, said product comprises at least one terpene selected from the group consisting of guaiol, limonene, pinene, borneol, linalool, terpinolene and combinations thereof, wherein each of said guaiol, limonene, pinene, borneol, linalool and terpinolene, when present is independently at a concentration of less than 5% by weight of the total terpene content.

According to an embodiment, the product comprises at least one herbal preparation selected from the group consisting of Valeriana officinalis, Viburnum opulus, Scutellaria lateriflora, Rosmarinus officinalis, Capsicum spp, Mentha piperita, Corydalis yanhusou, Paeonia lactiflora and combinations thereof, and the condition is selected from the group consisting of spasticity, muscle tension and combinations thereof. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol and combinations thereof.

According to an embodiment, the product comprises at least one herbal preparation selected from the group consisting of Boswellia spp, Galium aparine, Salix alba, Piscidia erythrina, Zanthoxylum spp, Corydalis yanhusou, Valeriana officinalis, Viburnum spp, Mentha piperita, Hypericum perforatum, Scutellaria laterifolia, Verbena officinalis and combinations thereof, and the condition comprises muscle pain and/or myalgia. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol and combinations thereof.

According to an embodiment, the product comprises at least one herbal preparation selected from the group consisting of Viburnum prunifolium, Zanthoxylum spp, Valeriana officinalis, Rosmarinus officinalis, Capsicum spp, Mentha piperita, Corydalis yanhusou, Paeonia lactiflora and combinations thereof, and the condition comprises spasms and/or muscle cramps. According to an embodiment, the product further comprises at least one primary terpene selected from the group consisting of myrcene, pinene, nerolidol, linalool, caryophyllene, limonene humulene, bisabolol, sabinene, citral, eucalyptol, terpineol, geraniol, fenchol and combinations thereof.

According to an embodiment, said product comprises at least one terpene selected from the group consisting of pinene, guaiol, linalool, borneol, terpineol and combinations thereof, wherein each of said pinene, guaiol, linalool, borneol and terpineol, when present is independently at a concentration of less than 5% by weight of the total terpene content.

According to an embodiment, the product comprises at least one herbal preparation selected from the group consisting of Viburnum prunifolium, Zanthoxylum spp, Piscidia erythrina, Boswellia spp, Capsicum spp, Valeriana officinalis, Scutellaria lateriflora, Rosmarinus officinalis, Mentha piperita, Corydalis yanhusou, Paeonia lactiflora and combinations thereof, and the condition comprises arm pain and/or leg pain. According to an embodiment, the product further comprises at least one primary terpene selected from the group consisting of linalool, terpinene, pinene, limonene, terpineol, myrcene, nerolidol and combinations thereof.

According to an embodiment, the product comprises at least one herbal preparation selected from the group consisting of Acorus calamus, Artemisia absinthium, Atractylodes macrocephala, Matricaria recutita, Zingiber officinalis, Bupleurum spp., Chamaemelum (Anthemis) nobile, Citrus reticulata, Curcuma longa, Ioscorea Villosa spp, Elettaria cardamomum, Foeniculum vulgare, Mentha piperita, Pimpinella anisum and combinations thereof, and the condition comprises nausea and/or vomiting. According to an embodiment, the product further comprises at least one primary terpene selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof.

According to an embodiment, the product comprises at least one herbal preparation selected from the group consisting of Acorus calamus, Arthmisia vulgaris, Atractylodes macrocephala, Angelica archangelica, Zingiber officinalis, Arctium lappa, Arthmisia vulgaris, Gentiana lutea spp and combinations thereof, and the condition is selected from the group consisting of lack of appetite, anorexia, cachexia and combinations thereof. According to an embodiment, the product further comprises at least one primary terpene selected from the group consisting of linalool, citronellol, geraniol, menthol, humulene, bisabolol, borneol, fenchol, linalool, limonene, terpineol, pinene, caryophyllene, myrcene, cymene, terpinene, valencene and combinations thereof.

According to an embodiment, said product comprises at least one terpene selected from the group consisting of pinene, guaiol, linalool, myrcene, terpinolene, terpineol and combinations thereof, wherein each of said pinene, guaiol, linalool, myrcene, terpinolene and terpineol, when present is independently at a concentration of less than 5% by weight of the total terpene content.

According to an embodiment, the product comprises at least one herbal preparation selected from the group consisting of Garcinia cambogia, Fucus vesiculosus, Gymnema Sylvestre, Iris versicolor, Capsicum spp, Cola vera\nitida, Coleus forskohlii, Foeniculum vulgare, Grifola frondosa Laminaria spp, Paullinia cupana and combinations thereof, and the condition comprises weight gain and/or excessive appetite. According to an embodiment, the product further comprises at least one primary terpene selected from the group consisting of eucalyptol, caryophyllene, limonene, humulene, phytol and combinations thereof.

According to an embodiment, the product comprises at least one herbal preparation selected from the group consisting of Curcuma longa, origanum, Filipendula ulmaria, Glycyrrhiza glabra, Phelodendron amurense, Boswellia spp, Matricaria recutita, Mentha piperita, Capsella bursa pastoris, Agrimonia eupatoria, Verbena officinalis, Ganoderma lucidum, Citri reticulata, Zingiber officinale and combinations thereof, and the condition comprises an inflammatory bowel disease. According to an embodiment, the product further comprises at least one primary terpene selected from the group consisting of limonene, caryophyllene, borneol, myrcene, pinene, terpinene, isopulegol, phellandrene, carene, carvacol, terpineol, humulene, linalool, nerolidol, guaiol and combinations thereof.

According to an embodiment, the product comprises at least one herbal preparation selected from the group consisting of Crataegus spp, Leonurus spp, Salvia miltiorrhiza, Ammi visnaga, Achillea millefolium, Allium sativum, Cimicifuga racemose, Olea europaea, Salvia miltiorrhiza, Tilia europea, Viburnum spp, Valeriana officinalis, Apium graveolens, Folia Taraxacum officinalis, Urtica dioica, Zea mays, Angelica sinensis, Capsicum spp, Cinnamomum zeylanicum, Eleutherococcus senticosus, Ginkgo biloba, Aesculus hippocastanum, Ruscus aculeatus and combinations thereof, and the condition comprises cardiovascular disease. According to an embodiment, the product further comprises at least one primary terpene selected from the group consisting of linalool, camphene, limonene, pinene, borneol and combinations thereof.

According to an embodiment, the product comprises at least one herbal preparation selected from the group consisting of Crataegus spp., Leonurus spp, Valeriana officinalis and combinations thereof, and the condition comprises palpitation and/or tachycardia. According to an embodiment, the product further comprises at least one primary terpene selected from the group consisting of linalool, camphene, limonene, pinene, borneol and combinations thereof.

According to an embodiment, the product comprises at least one herbal preparation selected from the group consisting of Bacopa monnieri, Ginkgo biloba, Centella asiatica, Rosmarinus officinalis, Rhodiola rosea, Rosmarinus officinalis, Curcuma longa, Eleutherococcus senticosus, Panax ginseng, Panax quinquefolius, Rhodiola rosea, Centella, Schisandra chinensis, Ganoderma lucidum, Withania somnifera and combinations thereof, and the condition comprises Alzheimer's disease. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Hypericum Perfortum, Rhodiola rosea, Valeriana officinalis, Ginkgo biloba, Viburnum sp and combinations thereof, and the condition comprises Parkinson's disease. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises Uncaria rhynchophylla, and the condition comprises Huntington's disease. According to an embodiment, the product further comprises at least one primary terpene selected from the group consisting of terpineol, caryophyllene, myrcene, terpineol, linalool, humulene, eucalyptol, pinene, pulegone, cymene, ocimene, limonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Uncaria rhynchophylla, Vibronum, Hypericum Perfortum, Rhodiola rosea, Valeriana officinalis, Ginkgo biloba and combinations thereof, and the condition comprises dystonia. According to an embodiment, the product further comprises at least one primary terpene selected from the group consisting of terpineol, caryophyllene, myrcene, terpineol, linalool, humulene, eucalyptol, pinene, pulegone, cymene, ocimenejimonene, nerolidol, sabinene, carene, citronellol, geraniol, menthol, borneol, terpinene, bisabolol, fenchol and combinations thereof.

According to an embodiment, the product comprises at least one herbal preparation selected from the group consisting of Hypericum perforatum, Viburnum spp, Verbena officinalis, Withania somnifera, Valeriana officinalis, Hypericum Perfortum, Rhodiola rosea, Valeriana officinalis, Ginkgo biloba and combinations thereof, and the condition comprises multiple sclerosis. According to an embodiment, the product further comprises at least one primary terpene selected from the group consisting of caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene, geraniol and combinations thereof.

According to an embodiment, the product comprises at least one herbal preparation selected from the group consisting of Astragalus membranceous, Ganoderma lucidum, Polygonum multiflorum, Ligustrum lucidum, Acorus gramineus and combinations thereof, and the condition comprises myasthenia gravis. According to an embodiment, the product further comprises at least one primary terpene selected from the group consisting of pinene, terpineol, eucalyptol, pulegone, cymene, ocimene, nerolidol, limonene, linalool, citronellol, geraniol, menthol, caryophyllene, borneol and combinations thereof.

According to an embodiment, the product comprises at least one herbal preparation selected from the group consisting of Bacopa monnierim, Cimicifuga racemosa, Ginkgo biloba, Paeonia lactiflora, Viscum album Uncaria rhynchophylla and combinations thereof, and the condition comprises epilepsy. According to an embodiment, the product further comprises at least one primary terpene selected from the group consisting of linalool, terpineol, ocimene, myrcene, limonene, caryophyllene and combinations thereof.

According to an embodiment, the product comprises at least one herbal preparation selected from the group consisting of Larrea mexicana, Grifola frondosa, Schisandra chinensis, Tabebuia impetiginos and Curcuma Longa, Ganoderma lucidum, Lentinula edodes, Trametes Versicolor Cordyceps sinensis, Panax ginseng, Astragalus membranaceus, Panax quinquefolius, (Silybum marianum, Scutellaria baicalensis, Boswellia spp, Glycyrrhiza glabra and combinations thereof and the condition comprises cancer and/or cancer-related symptoms. According to an embodiment, the product further comprises at least one primary terpene selected from the group consisting of myrcene, limonene, caryophyllene, caryophyllene oxide, terpinene, citronellol, linalool, geraniol, menthol, humulene, amyrin, cycloartenol, pinene, camphene, eucalyptol, terpineol, borneol, bisabolol, nerolidol, fenchol, guaiol and combinations thereof.

According to an embodiment, said product comprises at least one terpene selected from the group consisting of pinene, guaiol, linalool, myrcene, terpinolene, terpineol and combinations thereof, wherein each of said pinene, guaiol, linalool, myrcene, terpinolene and terpineol, when present is independently at a concentration of less than 5% by weight of the total terpene content.

According to an embodiment, the product comprises at least one herbal preparation selected from the group consisting of Ginkgo Biloba, Uncaria rhynchophylla and combinations thereof, and the therapeutic effect comprises stroke and/or traumatic brain injury. According to an embodiment, the product further comprises at least one primary terpene selected from the group consisting of myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol, carotol, bisabolol, guiaol and combinations thereof.

According to an embodiment, the product comprises at least one herbal preparation selected from the group consisting of Albizia lebbeck, Glycyrrhiza glabra, Sambucus nigra, Scutellaria baicalensis, Sophora flavescens, Echinacea spp, Inula helenium, Thymus vulgaris, Thuja occidentalis, Marrubium vulgare, Angelica archangelica, Drosera rotundifolia and combinations thereof, and the condition comprises an inflammatory airway disease. According to an embodiment, the product further comprises at least one primary terpene selected from the group consisting of pinene, eucalyptol, limonene, humulene, camphene, caryophyllene and combinations thereof.

According to an embodiment, the product comprises at least one herbal preparation selected from the group consisting of Echinacea spp., Ganoderma lucidum, Astragalus membranaceus, Rhodiola rosea, Panax ginseng, Eleutherococcus senticosus, Panax quinquefolius, Withania somnifera, Centella asiatica and combinations thereof, and the condition comprises a weakened immune system. According to an embodiment, product further comprises at least one primary terpene selected from the group consisting of linalool, pinene, borneol, caryophyllene, geraniol, amyrin, terpinene, myrcene, eucalyptol, humulene and combinations thereof.

According to an embodiment, the product comprises at least one herbal preparation selected from the group consisting of Curcuma Longa, Glycyrrhiza glabra Boswellia spp, Tanacetum parthenium, Harpagophytum procumbens, Bupleurum spp, Withania somnifera, Panax ginseng, Salix alba, Scutellaria baicalensis, Zingiber officinale and combinations thereof, and the condition comprises inflammation. According to an embodiment, the product further comprises at least one primary terpene selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, menth-2-en-1-ol, carotol, nerolidol and combinations thereof.

According to an embodiment, the product comprises at least one herbal preparation selected from the group consisting of Momordica charantia, Gymnema Sylvestre, Trigonella foenum graecum, Ganoderma lucidum, Panax quinquefolium, Panax ginseng, Withania somnifera, Astragalus membranaceus, Schisandra chinensis, Stevia rebaudiana, Curcuma longa, Ocimum sanctum, Silybum marianum, Olea europaea, Rehmania glutinosa, Eleutherococcus senticosus and combinations thereof, and the condition comprises diabetes. According to an embodiment, the product further comprises at least one primary terpene selected from the group consisting of limonene, pinene, linalool, humulene, myrcene, phytol, geraniol, bisabolol, borneol, guaiol, ocimene, fenchol, terpineol, terpinene, eucalyptol and combinations thereof.

According to an embodiment, the product comprises at least one herbal preparation selected from the group consisting of Boswellia spp, Galium aparine, Salix alba, Piscidia erythrina, Zanthoxylum spp, Corydalis yanhusou, Valeriana officinalis, Viburnum spp, Astragalus membranaceus, Glycyrrhiza glabra, Withania somnifera, Codonopsis pilosula, Eleutherococcus senticosus, Panax ginseng, Ganoderma lucidum, Centella asiatica, Schisandra chinensis, Atractylodes macrocephala, Lepidium meyenii, Rhodiola rosea, Hypericum perforatum, Scutellaria laterifolia, Verbena officinalis and combinations thereof, and the condition comprises fibromyalgia. According to an embodiment, the product further comprises at least one primary terpene selected from the group consisting of linalool, caryophyllene, limonene, humulene, eucalyptol, amyrin, cycloartenol, pinene, sabinene, myrcene, terpinene, borneol, bisabolol, guaiol, terpineol, ocimene, camphene and combinations thereof.

According to an embodiment, said product comprises at least one terpene selected from the group consisting of pinene, borneol, guaiol, linalool, terpineol, terpinolene and combinations thereof, wherein each of said pinene, borneol, guaiol, linalool, terpineol and terpinolene, when present is independently at a concentration of less than 5% by weight of the total terpene content.

According to an embodiment, the product comprises at least one herbal preparation selected from the group consisting of Anemarrhenae asphodeloides, Cimicifuga racemosa, Salvia officinalis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa and combinations thereof, and the condition comprises hot flashes. According to an embodiment, the product further comprises at least one primary terpene selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof.

According to an embodiment, the product comprises at least one herbal preparation selected from the group consisting of Anemarrhenae asphodeloides, Salvia officinalis, Schisandra chinensis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa, and the condition comprises facial flushing and/or night sweat. According to an embodiment, the product further comprises at least one primary terpene selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof.

According to an embodiment, the product comprises at least one herbal preparation selected from the group consisting of Apium graveolens, Barosma betulina, Hydrastis canadensis, Plantago spp, Equisitium arvense, Urtica dioica, Zea mays, Echinacea spp, Tabebuia impetiginosa, Phellodendron spp, Arctostaphylos uva ursi, Taraxacum officinalis folia, Capsella bursa pastoris, Phyllantus spp, Scutellaria baicalensis, Thuja occidentalis, Camellia sinensis, Althea officinalis, Glycyrrhiza glabra, Plantago spp and combinations thereof, and the condition comprises a lower urinary tract disorder.

According to an embodiment, the product comprises at least one herbal preparation selected from the group consisting of Withania somnifera, Vitex agnus castus, Dioscorea Villosa spp, Asparagus racemosa, Rehmania glutinosa, Angelica sinensis, Glycyrrhiza glabra, Trifolium pratense, Humulus lupulus, Medicago sativa, Cyperus rotundus and combinations thereof, and the condition comprises vaginal dryness.

According to an embodiment, the product comprises at least one herbal preparation selected from the group consisting of Bupleurum spp, Coptis chinensis, Echinacea spp, Cimicifuga racemosa, Anemarrhenae asphodeloides, Phytolacca americana, Tabebuia impetiginosa, Uncaria tormentosa, Andrographis paniculate, Berberis vulgaris, Hydrastis canadensis, Thymus vulgaris, Boswellia spp, Glycyrrhiza glabra, Curcuma longa, Achillea millefolium, Cimicifuga racemosa, Salix alba, Filipendula ulmaria, Calendula officinalis, Scutellaria baicalensis, Zingiber officinale, Tanacetum parthenium, Corydalis yanhusou, Valeriana officinalis, Viburnum spp, Galium aparine, Phytolacca americana, Iris versicolor, Calendula, Urtica, Zea mays and combinations thereof, and the condition comprises an infection of the urogenital or reproductive system.

According to an embodiment, the product comprises at least one herbal preparation selected from the group consisting of Caulophyllum thalictroides, Viburnum prunifolium, Vitex agnus castus, Cimicifuga racemosa, Angelica sinensis, Trifolium pratense, Lepidium meyenii, Salvia officinalis, Verbena officinalis, Corydalis yanhusou, Valeriana officinalis, Achillea millefolium, Capsella bursa pastoris, Agrimonia eupatoria, Calendula officinalis, Paeonia lactiflora and combinations thereof, and the condition is selected from the group consisting of fibroids, myomas and/or cysts in the female genital system and combinations thereof.

According to an embodiment, the product comprises at least one herbal preparation selected from the group consisting of Withania somnifera, Vitex agnus castus, Lepidium meyenii, Asparagus racemosa, Asphalentum ponjabianum-Mumio, Emblica officinalis Phylanthus emblica, Epimedium grandiflorum, Ganoderma lucidum, Panax ginseng, Rhodiola rosea, Tribulus terrestris, Turnera diffusa and combinations thereof, and the condition comprises reduced libido and/or sexual dysfunction.

According to an embodiment, said product comprises at least one primary terpene selected from the group consisting of limonene, linalool, myrcne, humulene, pinene, caryophyllene, bisabolol, guaiol, terpinolene, nerolidol, menthol and combinations thereof.

According to an embodiment, said product comprises at least one terpene selected from the group consisting of pinene, linalool, myrcene, terpineol and combinations thereof, wherein each of said pinene, linalool, myrcene and terpineol, when present is independently at a concentration of less than 5% by weight of the total terpene content.

According to an embodiment, the product comprises at least one herbal preparation selected from the group consisting of Vitex agnus castus, Cimicifuga racemosa, Angelica sinesis and combinations thereof, and the condition comprises Polycystic Ovary Syndrome (PCOS).

According to an embodiment, the product comprises at least one herbal preparation selected from the group consisting of Vitex agnus castus, Rehmania glutinosa Preparataangelica sinesis, Dioscorea Villosa spp, Aulophyllum thalictroides, Chamaelirium lutea, Cimicifuga (Actaea) racemosa, Cyperus rotundus and combinations thereof, and the condition is selected from the group consisting of amenorrhea and oligomenorrhea.

According to an embodiment, the product comprises at least one herbal preparation selected from the group consisting of Vitex agnus castus, Angelica sinesis, Pistacia lentiscus, Achillea millefolium, Aesculus hippocastanum, Agrimonia eupatoria, Alchemilla vulgaris, Calendula Officinalis, Capsella Bursa Pastoris, Chamaelirium lutea, Geranium maculatum, Hamamelis virginiana, Hydrastis canadensis, Pistacia lentiscus, Urtica dioica and combinations thereof, and the condition comprises hypermenorrhea.

According to an embodiment, the product comprises at least one herbal preparation is selected from the group consisting of Paeonia lactiflora, Viburnum prunifolium, Vitex Angelica sinesis, verbena officinalis, Scutalleria Laterifolia, Caulophyllum thalictroides, Chamaelirium lutea, Cimicifuga (Actaea) racemosa, Cyperus rotundus, Dioscorea Villosa spp and combinations thereof, and the condition comprises metrorrhagia.

According to an embodiment, the product comprises at least one herbal preparation selected from the group consisting of Vitex agnus castus, Angelica sinesis and combinations thereof, and the condition comprises premenstrual syndrome (PMS) and/or premenstrual dysphoric disorder (PMDD).

According to an embodiment, said product comprises at least one primary terpene selected from the group consisting of limonene, linalool, myrcne, humulene, pinene, caryophyllene, bisabolol, guaiol, terpinolene, nerolidol, menthol, camphene, citronellol, geraniol, fenchol and combinations thereof.

According to an embodiment, the product comprises at least one herbal preparation selected from the group consisting of Vitex agnus castus, Angelica sinesis, Cimicifuga racemosa, Withania somnifera, Chamaelirium lutea, Cyperus rotundus, Cypripedium pubescens, Leonurus spp, Curcuma Longa, Paeonia lactiflora, Iburnum prunifolium and combinations thereof, and the condition comprises a hormonal dysfunction and/or imbalance.

According to an embodiment, the product comprises at least one herbal preparation selected from the group consisting of Lepidium meyenii, Vitex agnus castus, Angelica sinesis, Chamaelirium lutea, Cimicifuga racemosa, Glycyrrhiza glabra, Dioscorea villosa, Trifolium pratense, Humulus lupulus, Leonurus cardiaca, Passiflora incarnata, Schisandra chinensis, Paeonia lactiflora, Angelica archangelica, Rehmania glutinosa, Urtica dioica and combination thereof, and the condition comprises a fertility disorder and/or infertility wherein the subject is a female.

According to an embodiment, the product comprises at least one herbal preparation selected from the group consisting of Chamaelirium lutea, Angelica sinesis, Cimicifuga racemosa, Tribulus terrestris, Serenoa serrulata, Lepidium meyenii, Withania somnifera, Serenoa serrulate, Ganoderma lucidum, Curcuma longa, Ginkgo biloba and combinations thereof, and the condition comprises a fertility disorder and/or infertility wherein the subject is a male.

According to an embodiment, the product comprises at least one herbal preparation selected from the group consisting of Caulophyllum thalictroides, Dioscorea Villosa spp, Rubus idaeus, Viburnum prunifolium, Panax ginseng, Zingiber officinale and combinations thereof, and the condition comprises pain associated with labour contractions.

According to an embodiment, the product comprises at least one herbal preparation selected from the group consisting of Cimicifuga racemosa, Paeonia lactiflora, Viburnum opulus, Vitex agnus castusm, Dioscorea villosa, Chamaelirium luteum, Trifolium pratense, Lepidium meyenii, Serenoa serullata, Salvia officinalis, Verbena officinalis, Humulus lupulus, Leonurus cardiaca, Achillea millefolium, Capsella bursa pastoris, Agrimonia, Curcuma longa, Schisandra chinensis, Cynara scolymus, Bupleurum spp, Silybum marianum, Mentha piperita, Corydalis yanhusou, Valeriana officinalis, Glycyrrhiza glabra, Salix alba, Filipendula ulmaria, Calendula officinalis, and combinations thereof, and the condition comprises endometriosis.

According to an embodiment, said wherein said product comprises at least one primary terpene selected from the group consisting of myrcene, linalool, caryophyllene, eucalyptol, nerolidol, limonene, nerolidol, sabinene, humulene, terpinolene, guaiol, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof.

According to an embodiment, said product comprises at least one selected from the group consisting of pinene, guaiol, linalool, borneol, humulene, terpinolene, myrcene and combinations thereof, wherein said pinene, guaiol, linalool, borneol, humulene, terpinolene and myrcene are each present at a concentration of less than 5% by weight of the total terpene content.

According to an embodiment, the product comprises at least one herbal preparation selected from the group consisting of Serenoa serrulata, Epimedium grandiflorum, Urtica dioica and combinations thereof, and the condition comprises a prostate abnormality.

According to an embodiment, the product comprises at least one herbal preparation selected from the group consisting of Humulus lupulus, Centella (Hydrocotyl) asiatica, Passiflora incarnata, Verbena officinalis, Valeriana officinalis, Bacopa monnieri, Avena sativa, Hypericum perforatum and combinations thereof, and the condition comprises autism and/or an autism spectrum disorder. According to an embodiment, the product further comprises at least one primary terpene selected from the group consisting of limonene, caryophyllene, pinene, myrcene, humulene, citronellol, eucalyptol, linalool, terpinen-4-ol, ocimene, geraniol, menthol, borneol, bisabolol, fenchol, guaoil and combinations thereof.

According to an embodiment, said product comprises at least one selected from the group consisting of linalool, myrcene, terpineol and combinations thereof, wherein each of said linalool, myrcene and terpineol, when present is at a concentration of less than 5% by weight of the total terpene content.

According to an embodiment, the product comprises at least one herbal preparation is selected from the group consisting of Bacopa monnieri, Centella (Hydrocotyl) asiatica, Humulus lupulus, Valeriana officinalis, Ginco Biloba, Lactuca spp, Nepeta cataria, Scutellaria lateriflora and combinations thereof, and the condition comprises attention deficit hyperactivity disorder (ADHD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Bacopa monnieri, Withania somnifer, Ginkgo biloba, Rosmarinus officinalis, Centella asiatica, Panax ginseng, Eleutherococcus senticosus, Ginkgo biloba, Rhodiola rosea, Hypericum perforatum, Scutellaria lateriflora, Zingiber officinale and combinations thereof, and the condition comprises attention deficit disorder (ADD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Hypericum perforatum, Rhodiola rosea, Valeriana officinalis, Passiflora incarnata, Withania somnifera and combinations thereof, and the condition comprises obsessive compulsive disorder (OCD). According to an embodiment, the product further comprises at least one primary terpene selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof and combinations thereof.

According to an embodiment, the product comprises at least one herbal preparation selected from the group consisting of Valeriana officinalis, Cypripedium pubescens, Passiflora incarnata, Hypericum perfuratum and combinations thereof, and the condition is selected from the group consisting of Tourette syndrome, motor tics, vocal tics and combinations thereof. According to an embodiment, the product further comprises at least one primary terpene selected from the group consisting of myrcene, linalool, limonene, pinene, nerolidol, caryophyllene, eucalyptol, geraniol, menthol, borneol, bisabolol, fenchol and combinations thereof.

According to an embodiment, the product comprises at least one herbal preparation selected from the group consisting of Drosera rotundifolia, Glycyrrhiza glabra, Plantago spp, Codonopsis pilosula, Althea officinalis, Glycyrrhiza glabra, Lactuca spp, Lonicera japonica, Lycium barbarum, Symphytum officinalis, Ulmus rubra\fulva, Verbascum Thapsus, Viola tricolor, Ziziphus jujuba and combinations thereof, and the condition comprises a dry cough. According to an embodiment, the product further comprises at least one primary terpene selected from the group consisting of pinene, eucalyptol, limonene and combinations thereof.

According to an embodiment, the product comprises at least one herbal preparation is selected from the group consisting of Thymus vulgaris, Inula helenium, Marrubium vulgare, Pimpinella anisum and combinations thereof, and the condition comprises a wet cough. According to an embodiment, the product further comprises at least one primary terpene selected from the group consisting of pinene, eucalyptol, limonene and combinations thereof.

According to an embodiment, the product comprises at least one herbal preparation selected from the group consisting of Glycyrrhiza glabra, Sambucus nigra, Scutellaria baicalensis, Ganoderma lucidum, Astragalus membranaceus, Stellaria media, Urtica dioica, Euphrasia officinalis, Ephedra sinica, Plantago spp and combinations thereof, and the condition comprises an allergy.

According to an embodiment, said product comprises at least one primary terpene selected from the group consisting of camphene, pinene, borneol, caryophyllene, geraniol and combinations thereof.

According to an embodiment, the product comprises at least one herbal preparation selected from the group consisting of Avena sativa, Lobelia inflate, Ganoderma lucidum, Withania somnifera and combinations thereof, and the condition comprises an addiction, including addiction to drugs, smoking, drinks, food, gambling, sex, and sports.

According to an embodiment, said product comprises at least one primary terpene selected from the group consisting of caryophyllene, pinene, myrcene, limonene, linalool, terpineol, citronellol, geraniol, humulene, bisabolol and combinations thereof.

According to an embodiment, the product comprises at least one herbal preparation comprise vitex and the condition comprises treatment of breast pain and/or mastalgia.

According to an embodiment, the administered product comprises a primary terpene and optionally at least one secondary terpenes, at least two, at least three secondary terpenes, at least four, or at least five secondary terpenes. As used herein, the term “primary terpene” refers to a terpene that forms at least 20% by weight of the total amount of terpenes in the product, at least 25%, at least 30%, at least 35%, at least 40%, at least 50%, at least 60%, at least 70% or at least 80%. As used herein, the term “primary terpene” refers to a terpene having the greatest amount in the composition. As used herein, the term “primary terpene” refers to a terpene that forms the highest terpene concentration in the composition. According to an embodiment, the product comprises multiple (e.g. two, three, or four) According to an embodiment, the primary terpene forms at least 20% by weight of the total terpene content, at least 30%, at least 40%, at least 50%, at least 60%, at least 70% or at least 80%, at least 90% by weight of the total terpene content. According to an embodiment, the product comprises multiple (e.g. two or three) terpenes, each one of which forms at least 20% by weight of the total amount of terpenes in the product and each one of these terpenes is considered a primary terpene. According to an embodiment, the product comprises multiple (e.g. two, three, or four) terpenes, each one of which forms at least 15% by weight of the total amount of terpenes in the product, at least 18%, at least 22%, at least 25%, at least 30%, at least 35%, or at least 40%, and each one of these terpenes is considered a primary terpene. As used here, in the term “secondary terpene” refers to a terpene that forms at least 10 parts per million (ppm) of the product. According to an embodiment, the content of the primary terpene in the product is at least 1.3 times greater than that of any secondary terpene, at least 1.5, at least 2, at least 2.2, at least 2.5, at least 3, at least 3.5, at least 4, at least 6, at least 8, at least 10, at least 15, at least 20, at least 25, or at least 30 times greater.

According to an embodiment, the primary terpene, the secondary terpenes or both are selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol, isomers thereof and combinations thereof. According to an embodiment, the primary terpene, the secondary terpenes or both, are selected from the group consisting of pinene, limonene, linalool, caryophyllene, myrcene, terpineol, eucalyptol, geraniol and combinations thereof. According to an embodiment, the primary terpene the secondary terpenes or both, are selected from the group consisting of pinene, limonene, linalool, caryophyllene, myrcene, terpineol, eucalyptol and combinations thereof. According to an embodiment, the primary terpene is selected from the group consisting of pinene, limonene, linalool, caryophyllene, myrcene, terpineol, eucalyptol, geraniol, humulene and combinations thereof. According to an embodiment, at least one of the terpenes is a-cyclic. According to an embodiment, at least one of the terpenes is cyclic. According to an embodiment, at least one of the terpenes is not found in Cannabis buds or is present there at less than 0.2%, less than 0.1%, less than 0.05% or less than 0.02%. Such terpene is referred to as “non-Cannabis terpene”. According to an embodiment, the terpene-enriched cannabinoid product comprises the non-Cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, terpenes form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, herbal preparation form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, the product comprises a composition, which is liquid at 30° C. According to an embodiment, the product comprises a composition, which is a suspension at 30° C. According to an embodiment, the product comprises a composition, which is essentially clear of haze or suspended solids at 30° C.

According to an embodiment, the product comprises Cannabis plant material. According to an embodiment, the product comprises Cannabis bud. According to an embodiment, the Cannabis bud forms at least 20% of the product, at least 30%, at least 40%, at least 50%, at least 60%, at least 70% or at least 80%.

According to an embodiment, the administered product is combines with at least one herbal extract, at least two, at least three, at least four, or at least five herbal extracts.

According to an embodiment, the administered product comprises less than 5% by weight glycol, less than 4%, less than 3%, less than 2%, or less than 1% by weight.

According to an embodiment, the administered product comprises less than 20% by weight water, less than 15% by weight, less than 14%, less than 13%, less than 12%, less than 11% or less than 10% by weight.

According to a related embodiment, the administered product comprises chlorophyll. According to an embodiment, the product comprises at least 0.5% by weight chlorophyll, at least 1%, at least 5%, at least 10%, at least 15%, at least 20% chlorophyll. According to an embodiment, the administered product comprises at least one flavonoid. According to an embodiment, the product comprises at least two, at least three, at least four, or at least five flavonoids. According to an embodiment, the product comprises at least one of bergamottin, apigenin, amentoflavone, quercetin and piperine. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine.

According to an embodiment, the administered product additionally contains an additive selected from the group consisting of antioxidants, emulsifiers and texturizers vegetable oils, plant extracts, honey, sucrose, glucose and fructose, ethanol, pharmaceutical excipients and combinations thereof.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the administrated product results in an increased and/or enhanced therapeutic effect compared with that of administrating a product comprising the same cannabinoid and optionally primary terpene amounts and being devoid of said herbal preparation. According to an embodiment, the product results in an increased and/or enhanced therapeutic effect compared with that of a product comprising the same cannabinoid and optionally primary terpene amounts and 10% the amount of said herbal extract, 20% the amount of said herbal extract, 30%, 40%, 50%, 60%, 70%, 80% or 90% of said herbal extract. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, said enhanced therapeutic effect comprises shorter onset time of the therapeutic effect. According to an embodiment, the cannabinoids, herbal preparation and optionally primary terpenes are present in specific amounts, and the onset time of the therapeutic effect, as measured by methods known in the art, is at least 20% shorter than that of a product comprising the same amounts of cannabinoids and primary terpenes and being devoid of said herbal preparation, at least 30%, at least 40%, at least 50%, at least 60%, or at least 70% shorter. Shorter onset time, or differently put an earlier effect, is important particularly in cases of sublingual, edible and topical delivery and in cases where a rapid effect is desired, as in treating pain. According to an embodiment, the cannabinoids, herbal preparation and optionally primary terpenes are present in specific amounts, and the onset time of the therapeutic effect, as measured by methods known in the art, is at least 20% shorter than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% shorter.

According to an embodiment, said enhanced therapeutic effect comprises longer onset time of the therapeutic effect. According to an embodiment, the cannabinoids, herbal preparation and optionally primary terpenes are present in specific amounts, and the onset time of the therapeutic effect, as measured by methods known in the art, is at least 20% longer than that of a product comprising the same amounts of cannabinoids and primary terpenes and being devoid of said herbal preparation, at least 30%, at least 40%, at least 50%, at least 60%, or at least 70% longer. According to an embodiment, products of delayed onset time are used in combination with shorter onset time to reach a sustained release effect. According to an embodiment, the cannabinoids, herbal preparation and optionally primary terpenes are present in specific amounts, and the onset time of the therapeutic effect, as measured by methods known in the art, is at least 20% longer than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% longer.

According to an embodiment, said enhanced therapeutic effect comprises greater and/or increased magnitude of the therapeutic effect. According to an embodiment, the cannabinoids, herbal preparation and optionally primary terpenes are present in specific amounts, and the magnitude of the therapeutic effect, as measured by methods known in the art, is at least 20% greater compared with that of a product comprising the same amounts of cannabinoids and primary terpenes and being devoid of said herbal preparation, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost. According to an embodiment, the cannabinoids, herbal preparation and optionally primary terpenes are present in specific amounts, and the magnitude of the therapeutic effect, as measured by methods known in the art, is at least 20% greater than that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% greater.

According to an embodiment, said enhanced therapeutic effect comprises longer and/or extended duration of the therapeutic effect. According to an embodiment, the cannabinoids, herbal preparation and optionally primary terpenes are present in specific amounts, and the duration of the therapeutic effect, as measured by methods known in the art, is at least 20% longer compared with that of a product comprising the same amounts of cannabinoids and primary terpenes and being devoid of said herbal preparation, at least 30%, at least 40%, at least 50%, at least 60%, at least 70%, at least 80%, at least 90% or at least 100% longer. According to an embodiment, the cannabinoids, herbal preparation and optionally primary terpenes are present in specific amounts, and the duration of the therapeutic effect, as measured by methods known in the art, is at least 20% longer compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% longer.

According to an embodiment, said enhanced therapeutic effect comprises smaller therapeutically effective amount of a product and/or reduced dosages of the product. According to an embodiment, the cannabinoids, herbal preparation and optionally primary terpenes are present in specific amounts, and the therapeutically effective amount of the product, as measured by methods known in the art, is at least 20% smaller compared with that of a product comprising the same amounts of cannabinoids and primary terpenes and being devoid of said herbal preparation, at least 30%, at least 40%, at least 50%, at least 60%, at least 70%, at least 80%, at least 90% or at least 100% smaller. According to an embodiment, the cannabinoids, herbal preparation and optionally primary terpenes are present in specific amounts, and the therapeutically effective amount of the product, as measured by methods known in the art, is at least 20% smaller compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises reduced secondary adverse symptoms. According to an embodiment, the cannabinoids, herbal preparation and optionally primary terpenes are present in specific amounts, and the secondary adverse symptoms, as measured by methods known in the art, are reduced by at least 20% compared with that of a product comprising the same amounts of cannabinoids and primary terpenes and being devoid of said herbal preparation by at least 30%, at least 40%, at least 50%, at least 60%, at least 70%, at least 80%, at least 90% or at least 100%. According to an embodiment, the cannabinoids, herbal preparation and optionally primary terpenes are present in specific amounts, and the secondary adverse symptoms, as measured by methods known in the art, are reduced by at least 20% compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced frequency of conditions and/or symptoms. According to an embodiment, the cannabinoids, herbal preparation and optionally primary terpenes are present in specific amounts, and the frequency of the conditions and/or symptoms, as measured by methods known in the art, is at least 20% smaller compared with that of a product comprising the same amounts of cannabinoids and primary terpenes and being devoid of said herbal preparation, at least 30%, at least 40%, at least 50% at least 60%, at least 70%, at least 80%, or at least 90% smaller. According to an embodiment, the cannabinoids, herbal preparation and optionally primary terpenes are present in specific amounts, and the frequency of the conditions and/or symptoms, as measured by methods known in the art is at least 20% smaller compared with that of a product amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced severity of conditions and/or symptoms. According to an embodiment, the cannabinoids, herbal preparation and optionally primary terpenes are present in specific amounts, and the severity of the conditions and/or symptoms, as measured by methods known in the art, is at least 20% smaller compared with that of a product comprising the same amounts of cannabinoids and primary terpenes and being devoid of said herbal preparation, at least 30%, at least 40%, at least 50% at least 60%, at least 70%, at least 80%, or at least 90% smaller. According to an embodiment, the cannabinoids, herbal preparation and optionally primary terpenes are present in specific amounts, and the severity of the conditions and/or symptoms, as measured by methods known in the art, is at least 20% smaller compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, said enhanced therapeutic effect comprises smaller and/or reduced consumption of other drugs. According to an embodiment, the cannabinoids, herbal preparation and optionally primary terpenes are present in specific amounts, and the consumption of other drugs is reduced by at least 20% compared with that of a product comprising the same amounts of cannabinoids and primary terpenes and being devoid of said herbal preparation, reduced by at least 30%, by at least 40%, by at least 50%, by at least 60%, by at least 70%, by at least 80%, or by at least 90%. According to an embodiment, the cannabinoids, herbal preparation and optionally primary terpenes are present in specific amounts, and the consumption of other drugs is reduced by at least 20% compared with that of a product comprising the same cannabinoid amount and 10% the amount of said herbal extract, 20% the amount of said herbal extract, 30%, 40%, 50%, 60%, 70%, 80% or 90% of said herbal extract, at least 30%, at least 40%, at least 50%, at least 60%, or at least 70% smaller.

According to an embodiment, the administrated product comprises tetrahydrocannabinol (THC) at a concentration of less than 5% by weight, and at least 0.5% by weight primary terpene. According to an embodiment, the product is applied to a child. According to an embodiment, the primary terpene is selected from the group consisting of limonene, linalool, eucalyptol, myrcene, caryophyllene, terpineol, bisabolol, pinene and combinations thereof. According to an embodiment, the composition comprises tetrahydrocannabinol (THC) in a concentration of less than 5%, less than 4%, less than 3%, less than 2%, or less than 1%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of limonene, linalool, myrcene and combination thereof. According to an embodiment, the herbal preparation is in a concentration of at least 0.5%, at least 1%, at least 5%, 10%, 20%, 30%, 40%, 50%, 60%, 70%, 80% or 90%. According to an embodiment, the composition comprises at least 2 of said herbal preparation, at least 3, at least 4, or at least 5.

According to an embodiment, the administrated product comprises CBD and optionally THC at CBD to THC weight/weight ratio greater than 1, wherein CBD concentration is less than 20% by weight and wherein primary terpene concentration is at least 0.5% by weight. According to an embodiment, the product is applied to a child. According to an embodiment, the primary terpene is selected from the group consisting of bisabolol, guaiol, linalool, terpineol, ocimene, humulene, caryophyllene, eucalyptol, pinene, limonene and combinations thereof. According to an embodiment, the product comprises CBD and optionally THC at CBD to THC weight/weight ratio greater 2, greater than 3, greater than 4 or greater than 5. According to an embodiment, the composition comprises cannabidiol (CBD) and/or cannabidiolic acid (CBDa) in a concentration of less than 18%, less than 16%, less than 14%, less than 12%, or less than 10%. According to an embodiment, the composition comprises cannabidiol (CBD) and/or cannabidiolic acid (CBDa), wherein CBD and/or CBDa concentration is of 10 to 20%, 10 to 15%, 15 to 20%, 12-18%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of linalool, terpineol, caryophyllene and combination thereof. According to an embodiment, the primary terpene is selected from the group consisting of linalool, terpineol, ocimene and combination thereof. According to an embodiment, the primary terpene is selected from the group consisting of eucalyptol, pinene, limonene and combination thereof. According to an embodiment, the primary terpene comprises caryophyllene and/or humulene. According to an embodiment, the herbal preparation is in a concentration of at least 0.5%, at least 1%, at least 5%, 10%, 20%, 30%, 40%, 50%, 60%, 70%, 80% or 90%. According to an embodiment, the composition comprises at least 2 of said herbal preparation, at least 3, at least 4, or at least 5.

According to an embodiment, the therapeutic effect is generated while administering the product in a vaporizer. According to an embodiment, the therapeutic effect is generated while administering the product by inhaling. According to an embodiment, the therapeutic effect is generated while administering the product by smoking. According to an embodiment, the therapeutic effect is generated while administering the product sublingually. According to an embodiment, the therapeutic effect is generated while applying the product, topically. According to an embodiment, the therapeutic effect is generated while administering the product in suppositories, including tampons and/or rectal candles. According to an embodiment, the therapeutic effect is generated while administering the product in a spray. According to an embodiment, the therapeutic effect is generated while administering the product as an edible, as a candy, as baked good, as a drink. According to an embodiment, the therapeutic effect is generated while administering the product as vaporizer liquid nasal preparation. According to an embodiment, the therapeutic effect is generated while administering the product as micro and nano-emulsions. According to an embodiment, the therapeutic effect is generated while administering the product as micro and nano-emulsions.

According to an embodiment, the enhanced therapeutic effect comprises reducing side effects associated with Cannabis consumption.

According to an embodiment, the method for treating a patient comprises administering to the patient for a first period of time a first herbal preparation-enriched Cannabis product comprising a first cannabinoid at a first cannabinoid amount and a first herbal preparation at a first primary terpene amount, followed by administering to the patient for a second period of time a second herbal preparation-enriched Cannabis product comprising the first cannabinoid amount and a second herbal preparation at a second herbal preparation amount. According to an embodiment, the method further comprises administering to the patient for a third period of time the first herbal preparation-enriched Cannabis product comprising the first cannabinoid amount and a third herbal preparation at a third primary terpene amount. According to an embodiment, the third herbal preparation is identical to the first primary terpene.

According to an embodiment, the method for treating a patient comprises administering to said subject for a first period of time a first said product comprising a first cannabinoid at a first cannabinoid amount and a first herbal preparation at a first herbal preparation amount, followed by; (i) administering to said subject for a second period of time said product comprising said first cannabinoid at said first cannabinoid amount or less and a second herbal preparation at a first herbal preparation amount; (ii) administering to said subject for a second period of time said product comprising said first cannabinoid at said first cannabinoid amount or less and a first herbal preparation at an increased first herbal preparation amount; (iii) administering to said subject for a second period of time said product comprising said first cannabinoid at said first cannabinoid amount or less, first herbal preparation at a first herbal preparation amount, and a second herbal preparation at a second herbal preparation amount; (iv) administering to said subject for a second period of time said product comprising said first cannabinoid at a said first cannabinoid amount or less, first herbal preparation at a first herbal preparation amount and a primary terpene at a selected amount; and/or (v) administering to said subject for a second period of time said product comprising said first cannabinoid at a said first cannabinoid amount or less, first herbal preparation at a first herbal preparation amount and at least one secondary terpene.

According to another embodiment, the method for treating a patient comprises administering to the patient a first herbal preparation-enriched Cannabis product comprising a first cannabinoid at a first cannabinoid amount and a first herbal preparation at a first herbal preparation amount and administering to the patient, at least 2 hours later a second herbal preparation-enriched Cannabis product comprising the first cannabinoid amount and a second herbal preparation at a second herbal preparation amount. According to another embodiment, the first herbal preparation Cannabis product is administered for day time and the second herbal preparation-enriched Cannabis product is administered for night time.

According to another embodiment, the method for treating a patient comprises administering to the patient (i) a first herbal preparation Cannabis product comprising a first cannabinoid at a first cannabinoid amount and a first herbal preparation at a first herbal preparation amount and (ii) a second herbal preparation Cannabis product comprising the first cannabinoid and a second herbal preparation a second herbal preparation amount.

According to an embodiment, the method for treating a patient comprises (i) administering to the patient multiple Cannabis products to find the best working one; (ii) mimicking the best working product by extracting Cannabis plant material to form an extract and blending the extract with suitable herbal preparations, whereby a mimicking product is formed and (iii) administering to the patient the mimicking product.

According to an embodiment, the method for treating a patient comprises administering to said patient a product selected from the group consisting of tablets, gel capsules, medical patches, topicals, creams, varnishes, sublingual oils, sprays, edibles, beverages, suppositories including tampons and/or rectal candles, cigarettes, vaporizer liquids, micro and nano-emulsions, preparations containing micro and nano-particle and combinations thereof, containing the administered product. According to an embodiment, the product comprising tablets containing the product. According to an embodiment, the product comprising gel capsules containing the product. According to an embodiment, the product comprising medical patches containing the product. According to an embodiment, the product comprising topicals containing the product. According to an embodiment, the product comprising creams containing the product. According to an embodiment, the product comprising varnishes containing the product. According to an embodiment, the product comprising sublingual oils containing the product. According to an embodiment, the product comprising edibles containing the product. According to an embodiment, the product comprising beverages containing the product. According to an embodiment, the product comprising tampons containing the product. According to an embodiment, the product comprising rectal candles containing the product. According to an embodiment, the product comprising cigarettes containing the product. According to an embodiment, the product comprising vaporizer liquids containing the product.

### Examples 1-100

The Table presents examples of compositions as described herein and exemplary uses thereof.

| | Cannab- | | | |
|---|---|---|---|---|
| | inoid | Herbal extract(s) | Primary terpene(s) | Indication(s) |
| 1 | CBN | Valeriana | myrcene, | Sleep disorder |
| | | officinalis, | caryophyllene, | Insomnia |
| | | Avena sativa, | eucalyptol, | |
| 2 | CBC and | Hypericum | Limonene, | Anxiety, |
| | CBD | Perfortum, | myrcene, | nervousness, |
| | | Passiflora, | terpinene, | restlessness |
| | | Humulus Lopus, | bisabolol, | |
| | | Whitania, | borneol | |
| | | Valeriana | | |
| | | officinalis | | |
| 3 | CBD | Hypericum | Limonene | Depression |
| | | Perfortum, | citronellol, | |
| | | Verbena | eucalyptol, | |
| | | Officinalis, | caryophyllene, | |
| | | Centella asiatica, | menth-2-en-1-ol | |
| | | Rhodiola rosea | | |
| 4 | CBD | Rhodiola rosea, | cymene, ocimene, | Difficulty in |
| | | Ginkgo biloba, | limonene, linalool, | concentrating, |
| | | Rosmarinus | humulene, carene | brain fog, |
| | | officinalis | | memory loss |
| 5 | CBD and | Lentinula edodes, | myrcene, terpineol, | Weakness, |
| | THC | Lepidium meyenii, | limonene, | reduced |
| | | Ocimum sanctum | borneol | mental |
| | | | | or physical |
| | | | | energy |
| 6 | CBD and | Withania | myrcene, terpineol, | Fatigue |
| | THC | somnifera, | limonene, | |
| | | Rhodiola rosea, | borneol | |
| | | Bacopa monnieri | | |
| 7 | CBD | Ginkgo biloba, | pinene, terpinene | Dizziness, |
| | | Lycium barbarum, | | defect |
| | | Rehmania | | in balance |
| | | glutinosa, | | |
| | | Bupleurum spp | | |
| 8 | THC + | Tanacetum | eucalyptol, | Headache, |
| | CBC | parthenium, | limonene, | migraine |
| | | Corydalis | nerolidol, | |
| | | yanhusuo, | fenchol, humulene | |
| | | Stachys betonica, | | |
| | | Piscidia erythrina | | |
| 9 | THC and | Mumio, Piscidia, | pinene, terpineol, | Neuropathic |
| | CBC | Rosmarinus | limonene, | pain |
| | | officinalis | geraniol, myrcene | |
| 10 | THC and | Origanum, | limonene, cymene, | Abdominal |
| | CBD | Matricaria recutita, | terpinene | Pain |
| | | Angelica | | |
| | | archangelica, | | |
| | | Filipendula ulmaria | | |
| 11 | CBC | Boswellia spp, | limonene, cymene, | Intestinal |
| | | Curcuma longa, | terpinene, | disorders, |
| | | Origanum, | germacrene, | gastroin- |
| | | Glycyrrhiza glabra, | geraniol, nerolidol, | testinal |
| | | Filipendula ulmaria | menthol, fenchol | pain |
| 12 | THC | Rheum officinalis, | geraniol, menthol, | Constipation |
| | | Juglans nigra, | fenchol | |
| | | Taraxacum | | |
| | | officinalis Radix | | |
| 13 | THC and | angelica sinesis, | limonene, cymene, | Pelvic pain |
| | CBD | piscidia, | citronellol, | |
| | | vibrunum opulus, | linalool, | |
| | | vitex | menthol, | |
| 14 | THC and | Boswellia spp, | myrcene, | Joint pain |
| | CBN | Curcuma longa, | caryophyllene, | (arthralgia), |
| | | Salix alba, | menth-2-en-1-ol, | joint stiffness, |
| | | Centella asiatica, | fenchol | arthritis |
| | | Galium aparine | | osteoarthritis |
| 15 | THC and | Valeriana | myrcene | Spasticity, |
| | CBD | officinalis, | humulene, | muscle |
| | | Viburnum opulus, | | tension |
| | | Scutellaria | | |
| | | lateriflora | | |
| 16 | THC and | Piscidia erythrina, | citral, eucalyptol, | Muscle pain, |
| | CBD | Zanthoxylum spp | geraniol | myalgia |
| 17 | CBD | Viburnum | myrcen, humulene, | Spasms, |
| | | prunifolium, | bisabolol, citral, | muscle |
| | | Zanthoxylum spp, | eucalyptol, | cramps |
| | | Valeriana | geraniol | |
| | | officinalis, | | |
| | | Scutellaria | | |
| | | lateriflora | | |
| 18 | THC | Viburnum | linalool, terpinene, | Arm pain, leg |
| | | prunifolium, | | pain |
| | | Zanthoxylum spp, | | |
| | | Piscidia erythrina, | | |
| 20 | THC | Acorns calamus, | | Nausea, |
| | | Artemisia | | vomiting |
| | | absinthium, | | |
| | | Atractylodes | | |
| | | macrocephala, | | |
| | | Matricaria recutita, | | |
| | | Zingiber officinalis | | |
| 21 | THC | Atractylodes | humulene, | Lack of |
| | | macrocephala, | bisabolol, | appetite, |
| | | Angelica | borneol, fenchol, | anorexia, |
| | | archangelica, | | cachexia |
| | | Zingiber officinalis | | |
| 22 | CBD | Gymnema | eucalyptol, | Weight gain, |
| | | Sylvestre, Iris | caryophyllene, | excessive |
| | | versicolor | limonene, | appetite |
| | | | humulene, | |
| | | | phytol | |
| 23 | THC and | Agrimonia | limonene, | Inflammatory |
| | CBC | eupatoria, Verbena | caryophyllene, | bowel disease |
| | | officinalis, | borneol, myrcene | |
| | | Ganoderma | | |
| | | lucidum, Citri | | |
| | | reticulata, Zingiber | | |
| | | officinale | | |
| 24 | CBD | Crataegus spp, | | Cardio- |
| | | Leonurus spp, | | vascular |
| | | Salvia miltiorrhiza, | | disease |
| | | Ammi visnaga | | |
| 25 | CBD | Crataegus spp., | linalool, camphene, | Palpitation, |
| | | Leonurus spp, | limonene, pinene, | tachycardia |
| | | Valeriana | borneol | |
| | | officinalis | | |
| 26 | CBD | Bacopa monnieri, | terpineol, | Alzheimer's |
| | | Ginkgo biloba, | pulegone | disease |
| | | Centella asiatica, | ocimene, limonene, | |
| | | Rosmarinus | | |
| | | officinalis, | | |
| | | Rhodiola rosea | | |
| 27 | THC and | Hypericum | terpineol, | Parkinson's |
| | CBC | Perfortum, | pulegone | disease |
| | | Rhodiola rosea, | ocimene | |
| | | Valeriana | mentholfenchol | |
| | | officinalis, | | |
| | | Ginkgo biloba | | |
| 28 | THC and | Uncaria | terpineol, | Huntingdon's |
| | CBC | rhynchophylla | ocimene, limonene, | disease |
| | | | sabinene, | |
| | | | mentholfenchol | |
| 29 | THC and | Uncaria | terpineol, | Dystonia |
| | CBD | rhynchophylla, | ocimene, limonene, , | |
| | | Vibronum | geraniol, | |
| | | | terpinene, | |
| 30 | THC and | Hypericum | caryophyllene, | Multiple |
| | CBD | perforatum, | myrcene, | sclerosis |
| | | Viburnum spp, | caryophyllene | |
| | | Verbena officinalis, | oxide, | |
| | | Withania | | |
| | | somnifera, | | |
| | | Valeriana | | |
| | | officinalis | | |
| 31 | THC | Astragalus | pinene, terpineol, | Myasthenia |
| | | membranceous, | caryophyllene, | gravis |
| | | Ganoderma | borneol | |
| | | lucidum, | | |
| | | Polygonum | | |
| | | multiflorum, | | |
| | | Ligustrum lucidum, | | |
| | | Acorns gramineus | | |
| 32 | CBD | Bacopa monnierim, | linalool | Epilepsy |
| | | Cimicifuga | | |
| | | racemosa, | | |
| | | Ginkgo biloba, | | |
| | | Paeonialactiflora, | | |
| | | Viscum album | | |
| | | Uncaria | | |
| | | rhynchophylla | | |
| 33 | THC and | Larrea mexicana, | myrcene, limonene, | Cancer, |
| | CBG | Grifola frondosa, | caryophyllene, | cancer- |
| | | Schisandra | caryophyllene | related |
| | | chinensis, | oxide, | symptoms |
| | | Tabebuia | terpinene, | |
| | | impetiginos and | | |
| | | Curcuma Longa | | |
| 34 | THC and | Ginkgo Biloba, | terpinene, | Stroke, |
| | CBD | Uncaria | caryophyllene, | traumatic |
| | | rhynchophylla | caryophyllene | brain |
| | | | oxide, | injury |
| | | | pinene, limonene, | |
| | | | humulene, | |
| 35 | THC | Albizia lebbeck, | pinene, eucalyptol, | Inflammatory |
| | | Glycyrrhiza glabra, | limonene, | airway disease |
| | | Sambucus nigra, | | |
| | | Scutellaria | | |
| | | baicalensis, | | |
| | | Sophora flavescens | | |
| 36 | CBD and | Echinacea spp., | geraniol, | Weakened |
| | CBG | Ganoderma | terpinene, myrcene, | immune |
| | | lucidum, | eucalyptol, | system |
| | | Astragalus | humulene | |
| | | membranaceus, | | |
| | | Rhodiola rosea | | |
| 37 | THC and | Curcuma Longa, | caryophyllene, | Inflammation |
| | CBN | Glycyrrhiza glabra | pinene, | |
| | | Boswellia spp | myrcene, | |
| | | | eucalyptol, linalool | |
| 38 | CBD | Momordica | myrcene, phytol, | Diabetes |
| | | charantia, | terpineol, terpinene, | |
| | | Gymnema | eucalyptol | |
| | | Sylvestre, | | |
| | | Trigonella foenium | | |
| | | graceum, | | |
| | | Ganoderma | | |
| | | lucidum | | |
| 39 | THC | Boswellia spp, | caryophyllene, | Fibromyalgia |
| | | Galium aparine, | sabinene, | |
| | | Salix alba, | myrcene, | |
| | | Piscidia erythrina, | | |
| | | Zanthoxylum spp | | |
| 40 | CBD | Anemarrhenae | caryophyllene, | Hot flashes |
| | | asphodeloides, | myrcene, | |
| | | Cimicifuga | terpinene, , | |
| | | racemosa, | fenchol | |
| | | Salvia officinalis, | | |
| | | Paeonialactiflora | | |
| 41 | CBD | Anemarrhenae | caryophyllene, | Facial |
| | | asphodeloides, | myrcene, | flushing, |
| | | Salvia officinalis, | fenchol | night sweat |
| | | Schisandra | | |
| | | chinensis, | | |
| | | Paeonia lactiflora | | |
| 42 | CBD and | Apium graveolens, | Pinene | Lower urinary |
| | CBG | Barosma betulina, | | tract disorder |
| | | Hydrastis | | |
| | | canadensis, | | |
| | | Plantago spp, | | |
| | | equisitium arvense, | | |
| | | Urtica dioica, Zea | | |
| | | mays | | |
| 43 | CBD and | Withania | | Vaginal |
| | CBG | somnifera, | | dryness |
| | | Vitex agnus castus, | | |
| | | Dioscorea Villosa | | |
| | | spp, | | |
| | | Asparagus | | |
| | | racemosa | | |
| 44 | CBD and | Bupleurum spp, | Eucalyptol, | Infection of |
| | CBC | Coptis chinensis, | humulene | the urogenital |
| | | Echinacea spp, | | or repro- |
| | | Cimicifuga | | ductive |
| | | racemosa, | | system |
| | | Anemarrhenae | | |
| | | asphodeloides | | |
| 45 | CBD and | Caulophyllum | | Fibroids, |
| | CBC | Thalictroides, | | myomas and/ |
| | | Viburnum | | or cysts in the |
| | | prunifolium, | | female genital |
| | | Vitex agnus castus | | system |
| 46 | THC | Withania | humulene, pinene, | Reduced |
| | | somnifera, | caryophyllene | libido, |
| | | Vitex agnus castus, | | sexual |
| | | Lepidium meyenii, | | dysfunction |
| | | Asparagus | | |
| | | racemosa | | |
| 47 | THC and | Vitex agnus castus, | | Polycystic |
| | CBD | Cimicifuga | | Ovary |
| | | racemosa, | | Syndrome |
| | | angelica sinesis | | |
| 48 | THC and | Vitex agnus castus, | | Amenorrhea, |
| | CBD | Rehmania | | oligomen- |
| | | glutinosa, | | orrhea |
| | | Prparata angelica | | |
| | | sinesis, | | |
| | | Dioscorea Villosa | | |
| | | spp | | |
| 49 | CBD and | Vitex agnus castus, | | Hypermen- |
| | CBC | Angelica sinesis, | | orrhea |
| | | Pistacialentiscus | | |
| 50 | CBD and | Paeonia lactiflora, | | Metrorrhagia |
| | CBC | Viburnum | | |
| | | prunifolium, | | |
| | | Vitex, Angelica | | |
| | | sinesis, | | |
| | | verbena officinalis, | | |
| | | scutalleria | | |
| | | Laterifolia | | |
| 51 | CBD and | Vitex agnus castus, | menthol, | Premenstrual |
| | CBC | Angelica sinesis | camphene, | syndrome, |
| | | | citronellol, | premenstrual |
| | | | geraniol | dysphoric |
| | | | | disorder |
| 52 | THC and | Vitex agnus castus, | | Hormonal |
| | CBD | angelica sinesis, | | dysfunction, |
| | | Cimicifuga | | hormonal |
| | | racemosa, | | inbalance |
| | | Withania somnifera | | |
| 53 | CBD | Lepidium meyenii, | | Female |
| | | Vitex agnus castus, | | fertility |
| | | Angelica sinesis, | | disorder |
| | | Chamaelirium | | and/or |
| | | lutea | | infertility |
| 54 | CBD | Chamaelirium | | Male fertility |
| | | lutea, | | disorder |
| | | Angelica sinesis, | | and/or |
| | | Cimicifuga | | infertility |
| | | racemosa, | | |
| | | Tribulus terrestris, | | |
| | | Serenoa serrulata | | |
| 55 | CBD and | Caulophyllum | Linalool, myrcene, | Pain |
| | THC | thalictroides, | pinene | associated |
| | | Dioscorea Villosa | | with labour |
| | | spp, | | contractions |
| | | Rubus idaeus, | | |
| | | Viburnum | | |
| | | prunifolium | | |
| 56 | CBD and | Cimicifuga | Linalool, myrcene, | Endometriosis |
| | THC | racemosa, | pinene, geraniol, | |
| | | Paeonia lactiflora, | humulene | |
| | | Viburnum opulus, | | |
| | | Vitex agnus | | |
| | | castusm | | |
| 57 | CBD and | Serenoa serrulata, | | Prostate |
| | THC | Epimedium | | abnormality |
| | | grandiflorum, | | |
| | | Urtica dioica | | |
| 58 | CBD | Humulus lupulus, | , | Autism and/or |
| | | Centella asiatica, | pinene, | autism |
| | | Passiflora incarnata, | humulene, | Spectrum |
| | | Verbena officinalis, | citronellol, | disorder |
| | | Valeriana | eucalyptol, | |
| | | officinalis, | borneol, | |
| | | Bacopa monnieri | | |
| 59 | CBD | Bacopa monnieri, | linalool, | Attention |
| | | Centella asiatica, | eucalyptol, | deficit |
| | | Humulus lupulus, | terpineol | hyperactivity |
| | | Valeriana | pinene, | disorder |
| | | officinalis, | | (ADHD). |
| | | Ginco Biloba | | |
| 60 | CBD | Bacopa monnieri, | linalool, eucalyptol, | Attention |
| | | Withania somnifer, | terpineol, myrcene, | deficit |
| | | Ginkgo biloba, | | disorder |
| | | Rosmarinus | | (ADD) |
| | | officinalis | | |
| 61 | THC and | Passiflora | linalool, eucalyptol, | Obsessive |
| | CBD | incarnata, | citronellol, | compulsive |
| | | Withania | menthol, | Disorder |
| | | somnifera | pinene, | (OCD) |
| 62 | THC | Valeriana | caryophyllene, | Tourette |
| | | officinalis, | eucalyptol, | syndrome, |
| | | Cypripedium | menthol, borneol, | motor tics, |
| | | pubescens, | | vocal tics |
| | | Passiflora | | |
| | | incarnata | | |
| 63 | CBD and | Scutellaria | | Allergy |
| | CBG | baicalensis, | | |
| | | Ganoderma | | |
| | | lucidum, | | |
| | | astragalus | | |
| | | membranaceus, | | |
| | | Stellaria media | | |
| 64 | CBD | Avena sativa, | | Addiction |
| | | Lobelia inflate, | | |
| | | Ganoderma | | |
| | | lucidum, | | |
| | | Withania | | |
| | | somnifera | | |
| 65 | THC and | Vitex | | Breast pain, |
| | CBN | | | mastalgia |

### Example 68: Treating an Elderly Man with Osteoarthritis

A man at the age of 75 diagnosed with osteoarthritis is treated by administering ground Cannabis in a vaporizer, together with a herbal preparation prepared from Boswellia spp. The ground Cannabis contains 12% THC, 0.5% CBD and a primary terpene containing humulene. Administrating involves 2 puffs per dose, twice a day. Each administrated dose contains 15 mg of THC, 0.6 mg of CBD, 6 mg humulene and 20 mg Boswellia spp extract.

### Example 69: Treating an Elderly Man with Alzheimer Disease

A man at the age of 82 diagnosed with Alzheimer disease is treated by administering Cannabis oil sublingually together with a herbal preparation prepared from Bacopa monnieri, and Ginkgo biloba. The oil contains 4% THC, 12% CBD and a primary terpene containing terpineol. Administrating involves 4 droplets per dose, twice per day. Each administrated dose contains 6 mg of THC, 19 mg of CBD, 4 mg terpineol and 16 mg of said herbal preparation.

### Example 70: Treating a Woman with Multiple Sclerosis

A woman at the age of 25 diagnosed with multiple sclerosis and presenting with symptoms of spasticity is treated by administering ground Cannabis in a vaporizer, together with a herbal preparation prepared from Hypericum perforatum, Viburnum spp. and Verbena officinalis. The ground Cannabis contains 8% THC, 5% CBD and a primary terpene containing pinene. Administrating involves 10 puffs per dose, twice a day. Each administrated dose contains 50 mg of THC, 31 mg of CBD, 15 mg pinene and 30 mg of said herbal preparation.

### Example 71: Treating a Child with ADD

A child at the age of 12 diagnosed with ADHD is treated by administering Cannabis oil sublingually, together with a herbal preparation prepared from Bacopa monnieri. The Cannabis oil contains 0.2% THC, 18% CBD and a primary terpene containing eucalyptol. Administrating involves 1 droplet per dose, twice a day. Each administrated dose contains 0.1 mg of THC, 8 mg of CBD, 5 mg eucalyptol and 50 mg of said herbal preparation.

### Example 72: Treating an Elderly Woman with Dementia

A woman at the age of 70 with memory loss is treated by administering Cannabis tablets together with a herbal preparation prepared from Bacopa monnieri, Centella asiatica, Rhodiola rosea, Ginkgo biloba and Rosmarinus officinalis. The tablets contain 2% THC, 6% CBD and a primary terpene containing pinene. Administrating involves 2 tablets per dose, twice a day. Each administrated dose contains 3 mg of THC, 9 mg of CBD, 4 mg pinene and 40 mg of said herbal preparation.

### Example 73: Treating a Woman with Myalgia

A woman at the age of 58 with symptoms of myalgia is treated by administering ground Cannabis in a vaporizer, together with a herbal preparation prepared from Boswellia spp, Galium aparine and Salix alba. The ground Cannabis contains 15% THC, 1% CBD and a primary terpene containing sabinene. Administrating involves 3 puffs per dose, 3 times per day. Each administrated dose contains 30 mg of THC, 2 mg of CBD, 12 mg sabinene and 24 mg of said herbal preparation.

### Example 74: Treating a Woman with Symptoms of Insomnia

A woman at the age of 48 with symptoms of insomnia is treated by administering Cannabis cigarettes together with a herbal preparation prepared from Valeriana officinalis, Avena sativa, Passiflora incarnate and Humulus Lopulus. The cigarette contains 16% THC, 3% CBD and a primary terpene containing terpinene. Administrating involves 5 puffs per dose, 30 minutes before bedtime. Each administrated dose contains 50 mg of THC, 9 mg of CBD, 10 mg terpinene and 20 mg of said herbal preparation.

### Example 75: Treating a Woman with Gastrointestinal Pain

A woman at the age of 33 with symptoms of gastrointestinal pain is treated by administering Cannabis oil sublingually, together with a herbal preparation prepared from Glycyrrhiza glabra. The Cannabis oil contains 5% THC, 10% CBD and a primary terpene containing terpineol. Administrating involves 1 droplet per dose, twice a day. Each administrated dose contains 2 mg of THC, 4 mg of CBD, 2 mg terpineol and 4 mg of said herbal preparation.

### Example 76: Treating a Child with Tourette Syndrome

A child at the age of 10 diagnosed with Tourette syndrome is treated by administering ground Cannabis in a vaporizer, together with a herbal preparation prepared from Valeriana officinalis, Cypripedium pubescens and Passiflora incarnata. The ground Cannabis contains 9% THC and 6% CBD. Administrating involves 3 puffs per dose, 3 times per day. Each administrated dose contains 17 mg of THC, 11 mg of CBD and 30 mg of said herbal preparation.

### Example 77: Treating a Woman with Symptoms of Anxiety and Stress

A woman at the age of 51 with symptoms of anxiety and stress is treated by administering tablets of Cannabis extract together with a herbal preparation prepared from Hypericum Perfortum. Each tablet contains 0.5% THC and 16% CBD. Administrating involves 1 tablet per dose, twice a day. Each administrated dose contains less than 0.4 mg of THC, 13 mg of CBD and 20 mg of said herbal preparation.

### Example 78: Treating a Child with Epilepsy

A child at the age of 5 diagnosed with severe epilepsy is treated by administering Cannabis oil sublingually, together with a herbal preparation prepared from Bacopa monnierim, Cimicifuga racemosa, Ginkgo biloba, Paeonia lactiflora, Viscum album and Uncaria rhynchophylla. The Cannabis oil contains 0.1% THC, 18% CBD and a primary terpene containing linalool. Administrating involves 3 droplets per dose, twice a day. Each administrated dose contains 0.1 mg of THC, 21.5 mg of CBD, 8 mg linalool and 20 mg of said herbal preparation.

### Example 79: Treating a Woman with OCD

A woman at the age of 28 diagnosed with OCD is treated by administering Cannabis oil sublingually, together with a herbal preparation prepared from Hypericum perforatum and Rhodiola rosea. The Cannabis oil contains 12.5% THC, 12.5% CBD and a primary terpene containing geraniol. Administrating involves 1 droplet per dose, 3 times per day. Each administrated dose contains 5 mg of THC, 5 mg of CBD, 3 mg geraniol and 10 mg of said herbal preparation.

### Example 80: Treating a Woman with Neuropathic Pain

A woman at the age of 33 diagnosed with multiple sclerosis and presenting with neuropathic pain is treated by administering Cannabis oil sublingually, together with a herbal preparation prepared from Mumio, Piscidia and Rosmarinus officinalis. The Cannabis oil contains 10% THC and 2% CBD. Administrating involves 1 droplet per dose, twice a day. Each administrated dose contains 4 mg of THC, 0.8 mg of CBD and 25 mg of said herbal preparation.

### Example 81: Treating a Woman with Crohn's Disease

A woman at the age of 30 diagnosed with Crohn's disease treated by administering tablets of Cannabis extract, together with a herbal preparation prepared from Curcuma longa. Each tablet contains 1.3% THC, 31% CBD and a primary terpene containing pinene. Administrating involves 1 tablet per dose, twice a day. Each administrated dose contains 0.5 mg of THC, 12.5 mg of CBD, 2.5 mg pinene and 25 mg of said herbal preparation.

### Example 82: Treating a Woman with Symptoms of Depression

A woman at the age of 25 with symptoms of depression is treated by administering Cannabis cigarettes, together with a herbal preparation prepared from Hypericum Perfortum, Verbena Officinalis and Centella asiatic. The cigarettes contain 0.5% THC and 15% CBD. Administrating involves 5 puffs per dose, 3 times per day. Each administrated dose contains 1.5 mg of THC, 45 mg of CBD and 10 mg of said herbal preparation.

### Example 83: Treating an Elderly Man with Parkinson Disease

A man at the age of 68 diagnosed with Parkinson disease presenting with symptoms of severe spasticity is treated by administering ground Cannabis in a vaporizer, together with a herbal preparation prepared from Hypericum Perfortum, Rhodiola rosea and Valeriana officinalis. The ground Cannabis contains 13.5% THC, 0.4% CBD and a primary terpene containing limonene. Administrating involves 3 puffs per dose, twice a day. Each administrated dose contains 25 mg of THC, 0.75 mg of CBD, 8 mg limonene and 15 mg of said herbal preparation.

### Example 84: Treating an Elderly Woman with Dystonia

A woman at the age of 72 diagnosed with dystonia is treated by administering Cannabis oil sublingually, together with a herbal preparation prepared from Uncaria rhynchophylla, and Vibronum. The Cannabis oil contains 5.5% THC, 5.5% CBD and a primary terpene containing pinene. Administrating involves 4 droplet per dose, 3 times per day. Each administrated dose contains 9 mg of THC, 9 mg of CBD, 4 mg pinene and 40 mg of said herbal preparation.

### Example 85: Treating a Woman with Symptoms of Nausea and Vomiting

A woman at the age of 25 with symptoms of nausea and vomiting is treated by administering Cannabis oil sublingually together with a herbal preparation prepared from Acorus calamus, Artemisia absinthium, Atractylodes macrocephala, Matricaria recutita and Zingiber officinalis. The Cannabis oil contains 2% THC and 12% CBD. Administrating involves 3 droplets per dose, twice a day. Each administrated dose contains 2.5 mg of THC, 14.5 mg of CBD and 0 mg of said herbal preparation.

### Example 86: Treating a Man with Symptoms of Nervousness and Irritability

A man at the age of 49 with symptoms of nervousness and irritability is treated by administering Cannabis oil sublingually together with a herbal preparation prepared from Hypericum Perfortum. The oil contains 5.5% THC, 9% CBD and a primary terpene containing imonene. Administrating involves 5 droplets per dose, 3 times per day. Each administrated dose contains 11 mg of THC, 18 mg of CBD, 10 mg imonene and 20 mg of said herbal preparation.

### Example 125: Treating an Elderly Man with Diabetes

A man at the age of 71 diagnosed with diabetes is treated by administering Cannabis oil sublingually together with a herbal preparation prepared from Momordica charantia. The oil contains 8% THC, 3% CBD and a primary terpene containing bisabolol. Administrating involves 4 droplets per dose, 3 times per day. Each administrated dose contains 13 mg of THC, 5 mg of CBD and 4 mg bisabolol.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the claimed embodiments.

What is claimed is:

1. A method of treatment comprising administering to a subject in need a therapeutically effective amount of a product comprising:
(i) at least one cannabinoid;
(ii) at least one herbal preparation, wherein said herbal preparation is produced by at least one of distilling and extracting a portion of a plant, wherein said plant is not Cannabis sativa;
(iii) at least one primary terpene selected from the group consisting of caryophyllene, linalool, pinene, eucalyptol, limonene, guaiol, geraniol, sabinene, terpineol, bisabolol, humulene, myrcene, nerolidol and combinations thereof;
(iv) optionally at least three secondary terpenes; and
(v) at least 5% by weight of a non-cannabinoid, non-terpene carrier.

2. The method of claim 1, wherein said primary terpene is present at a concentration of at least 5% of a concentration of said herbal preparation.

3. The method of claim 1, wherein said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, Origanum, Paeonia lactiflora, Panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, Vibronum, Viburnum opulus, Viburnum prunifolium, Viscum Albumvitex, Vitex agnus castus, Witania, Zanthoxylum spp, Zingiber officinalis and combinations thereof.

4. The method of claim 1, wherein said product comprises a homogenous preparation of Cannabis flowers and herbal preparation.

5. The method of claim 1, wherein the treatment is of at least one condition and/or symptom associated therewith, wherein said condition is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

6. The method of claim 1, wherein said at least one cannabinoid comprises tetrahydrocannabinol (THC) at a concentration of less than 5% by weight, and at least 0.5% by weight herbal preparation.

7. The method of claim 1, wherein said at least one herbal preparation is selected from the group consisting of Valeriana officinalis, Avena sativa, Passiflora incarnate, Humulus Lopulus, Bacopa monnieri, Hypericum perforatum, Lavendula officinalis, Matricaria recutita, Melissa officinalis, Piscidia erythrina, Scutellaria lateriflora, Ziziphus jujuba, Zizyphus spinosa, Lactuca spp. and combinations thereof, and wherein said treatment is of a sleep disorder or insomnia.

8. The method of claim 1, wherein said at least one herbal preparation is selected from the group consisting of Hypericum Perfortum, Passiflora, Humulus Lopus, Whitania and Valeriana officinalis, Bacopa monnieri, Avena sativa, Centella (Hydrocotyl) asiatica, Cypripedium pubescens, Ganoderma lucidum, Lactuca spp. Leonurus spp. Melissa officinalis Rhodiola rosea, Scutellaria lateriflora, Tilia europea, Turnera diffusa, Ziziphus jujuba, Zizyphus spinosa and wherein the treatment is of a condition selected from the group consisting of anxiety, nervousness, restlessness, stress, mood swing, affect disorder and combinations thereof.

9. The method of claim 1, wherein said at least one herbal preparation is selected from the group consisting of Boswellia spp, Curcuma longa, Origanum and Glycyrrhiza glabra, Filipendula ulmaria, Matricaria recutita, Angelica archangelica, Foeniculum vulgare, Mentha piperita, Zingiber officinale, Glycyrrhiza glabra, Angelica archangelica and combinations thereof, and wherein said treatment is of a condition selected from the group consisting of an intestinal disorder, gastrointestinal pain and a combination thereof.

10. The method of claim 1, wherein said at least one herbal preparation is selected from the group consisting of Angelica sinesis, Piscidia, Vibrunum opulus, Vitex agnus castus, Dioscorea villosa, Cimicifuga racemosa, Trifolium pretense, Capsella bursa pastoris, Achillea millefolium, Boswellia spp, Paeonia lactiflora, and combinations thereof, and wherein said treatment is of pelvic pain.

11. The method of claim 1, wherein said at least one herbal preparation is selected from the group consisting of Viburnum prunifolium, Zanthoxylum spp, Valeriana officinalis, Rosmarinus officinalis, Capsicum spp, Mentha piperita, Corydalis yanhusou, Paeonia lactiflora and combinations thereof, and wherein said treatment is of a condition selected from the group consisting of spasms, muscle cramps and a combination thereof.

12. The method of claim 1, wherein said at least one herbal preparation is selected from the group consisting of Curcuma longa, origanum, Filipendula ulmaria, Glycyrrhiza glabra, Phelodendron amurense, Boswellia spp, Matricaria recutita, Mentha piperita, Capsella bursa pastoris, Agrimonia eupatoria, Verbena officinalis, Ganoderma lucidum, Citri reticulata, Zingiber officinale and combinations thereof, and wherein said treatment is of an inflammatory bowel disease.

13. The method of claim 1, wherein said at least one herbal preparation is selected from the group consisting of Anemarrhenae asphodeloides, Cimicifuga racemosa, Salvia officinalis, Paeonia lactiflora, Vitex agnus castus, Angelica sinensis, Dioscorea villosa, Glycyrrhiza glabra, Trifolium pratense, Lepidium meyenii, Salvia officinalis, Verbena officinalis, Humulus lupulus, Leonurus cardiaca, Rehmania glutinosa and combinations thereof, and wherein said treatment is of hot flashes.

14. The method of claim 1, wherein said at least one herbal preparation is selected from the group consisting of Vitex agnus castus, Cimicifuga racemosa, Angelica sinesis and combinations thereof, and wherein said treatment is of Polycystic Ovary Syndrome (PCOS).

15. The method of claim 1, wherein said at least one herbal preparation is selected from the group consisting of Vitex agnus castus, Rehmania glutinosa Preparataangelica sinesis, Dioscorea Villosa spp, Aulophyllum thalictroides, Chamaelirium lutea, Cimicifuga (Actaea) racemosa, Cyperus rotundus and combinations thereof, and wherein said treatment is of a condition selected from the group consisting of amenorrhea and oligomenorrhea.

16. The method of claim 1, wherein said at least one herbal preparation is selected from the group consisting of Vitex agnus castus, Angelica sinesis, Cimicifuga racemosa, Withania somnifera, Chamaelirium lutea, Cyperus rotundus, Cypripedium pubescens, Leonurus spp, Curcuma Longa, Paeonia lactiflora, Iburnum prunifolium and combinations thereof, and wherein said treatment is of a condition selected from the group consisting of a hormonal dysfunction, a hormonal imbalance and a combination thereof.

17. The method of claim 1, wherein said at least one herbal preparation is selected from the group consisting of Cimicifuga racemosa, Paeonia lactiflora, Viburnum opulus, Vitex agnus castusm, Dioscorea villosa, Chamaelirium luteum, Trifolium pratense, Lepidium meyenii, Serenoa serullata, Salvia officinalis, Verbena officinalis, Humulus lupulus, Leonurus cardiaca, Achillea millefolium, Capsella bursa pastoris, Agrimonia, Curcuma longa, Schisandra chinensis, Cynara scolymus, Bupleurum spp, Silybum marianum, Mentha piperita, Corydalis yanhusou, Valeriana officinalis, Glycyrrhiza glabra, Salix alba, Filipendula ulmaria, Calendula officinalis and combinations thereof, and wherein said treatment is of endometriosis.

18. The method of claim 1, wherein said at least one herbal preparation is selected from the group consisting of Serenoa serrulata, Epimedium grandiflorum, Urtica dioica and combinations thereof, and wherein said treatment is of a prostate abnormality.

19. The method of claim 1, wherein said at least one herbal preparation is selected from the group consisting of Humulus lupulus, Centella (Hydrocotyl) asiatica, Passiflora incarnata, Verbena officinalis, Valeriana officinalis, Bacopa monnieri, Avena sativa, Hypericum perforatum and combinations thereof, and wherein said treatment is of autism or an autism spectrum disorder.

20. The method of claim 1, wherein said at least one herbal preparation is selected from the group consisting of Valeriana officinalis, Cypripedium pubescens, Passiflora incarnata, Hypericum perfuratum and combinations thereof, and wherein said treatment is of a condition selected from the group consisting of Tourette syndrome, motor tics, vocal tics and combinations thereof.

21. The method of claim 1, wherein said at least one herbal preparation is selected from the group consisting of Glycyrrhiza glabra, Sambucus nigra, Scutellaria baicalensis, Ganoderma lucidum, Astragalus membranaceus, Stellaria media, Urtica dioica, Euphrasia officinalis, Ephedra sinica, Plantago spp and combinations thereof, and wherein said treatment is of an allergy.
